SEQUENCE LISTING (32 sequences)

SEQ ID NO: 1 - DNA, 50 bp, Plasmid pUC18
cgaaagggcctcgtgatacgtaggttaatgtcatgataataatggtttct

SEQ ID NO: 2 - DNA, 50 bp, M13 lambda phage DNA
agggttttcccagtcacgacgttgtaaaacgacggccagtgccaagcttg

SEQ ID NO: 3 - DNA, 70 bp, Homo sapiens
acataatttacacgaaagcaatgctatcacctcccctgtgtggacttgggagaggactgggccattctcc

SEQ ID NO: 4 - DNA, 70 bp, Homo sapiens
ggagggggccttggcatcttctctttatgtctctgagctgtgccttcgccaccccttctgggtcactcag

SEQ ID NO: 5 - DNA, 70 bp, Homo sapiens
tctcttattgactgttgtgcatgctgtggtgctttgaggtaggtctggtgaaggtccatgagacaaggct

SEQ ID NO: 6 - DNA, 70 bp, Homo sapiens
ccagtagtgcaaatgcatgaagggctctcactgttggtagtcattcagcaataaatagtaagtccacgcc

SEQ ID NO: 7 - DNA, 70 bp, Homo sapiens
tcgggcagctgcttgaacaagttccgcagggtgctgagctcgcgcgacagctgctccaccttcttctgca

SEQ ID NO: 8 - DNA, 70 bp, Homo sapiens
gttttcttacattctggagaaccgaagctcagctcagccctcttccttattttgctcccaaagcctcccc

SEQ ID NO: 9 - DNA, 70 bp, Homo sapiens
ggcaacccatttacatgcagccttgtaacatttgtctacatcacacaaggcgaccaaggacacttccaac

SEQ ID NO: 10 - DNA, 70 bp, Homo sapiens
aggcctggctcaacatgctactaactaccagctctctgaagtgtcactgggaacaatacacactccatgc

SEQ ID NO: 11 - DNA, 69 bp, Homo sapiens
ggttgagcacagggtactttattgatggtacatgacaaggtgcggctccctaggcccctcccctcttca

SEQ ID NO: 12 - DNA, 70 bp, Homo sapiens
gcatgtaggcctagaaaaaggctctctgaaaccctcaatggcaactggtgaacggtaacactgattgccc

SEQ ID NO: 13 - DNA, 70 bp, Homo sapiens
acacacaacccatgggatcttgcttaggttggctgcctagttggcccctgagataaagccttgtaatcac

SEQ ID NO: 14 - DNA, 70 bp, Homo sapiens
gtcaaaggaagttcacggggaactaggaatgtgtcttcttcctccagaattcaacccttggaagatgggg

SEQ ID NO: 15 - DNA, 70 bp, Homo sapiens
cagcagtaaatgctccagttgtagctgtgttttctttgttttctttgtcgaacttgaagtaggtgcactg

SEQ ID NO: 16 - DNA, 70 bp, Homo sapiens
cttctcaggaaacgtacgcattgatttgcaccttgtgtgtccacctgtaaacatcaaattagtgcaggcc

SEQ ID NO: 17 - DNA, 70 bp, Homo sapiens
ctgaccagaagaaggaatgcccattaacaacaacaatctgaggtgcccatgctacatttgccgaagagcc

SEQ ID NO: 18 - DNA, 70 bp, Homo sapiens
gcactaccaacacagctggcaatgacaagactgggagtatcaaactaggattgttagttcaattaaaact

SEQ ID NO: 19 - DNA, 70 bp, Homo sapiens
cactgcaaccccccttcctccagcctcctgaaacatcgcactagcctttggtaagcaattccatatagat

SEQ ID NO: 20 - DNA, 70 bp, Homo sapiens
ggggccagctccgccgcgatcgccccctcttcccctccctgttaaatacacaaatatattatattcaata

SEQ ID NO: 21 - DNA, 70 bp, Homo sapiens
ggcgtaacgagactttactgaaaacagaaaaccgggcgaaccaaggattacaaacaggaatgtggactcg

SEQ ID NO: 22 - DNA, 70 bp, Homo sapiens
accaccctctctgggccctttcattctctgctatggactgagtggaccagcttggatcaaaatcctcaaa

SEQ ID NO: 23 - DNA, 70 bp, Homo sapiens
gcttttgctcctctgcttggacggacaggatgtatgctgtggcttttttaaggataactaccttgggggc

SEQ ID NO: 24 - DNA, 70 bp, Homo sapiens
agttaaatcgagaatgattcaggcgggccggctctctgagcacctttggatgcacttcagcttctgtctt

SEQ ID NO: 25 - DNA, 70 bp, Homo sapiens
gtcgaagaacacggtggggttgaccatggctaatagtacacggttttcctcggcggtggcgtctgcaaaa

SEQ ID NO: 26 - DNA, 70 bp, Homo sapiens
ccaatacaggcgctctgctgtctccgcttccactccactagttcatcattaatcagggcattctgggtaa

SEQ ID NO: 27 - DNA, 70 bp, Homo sapiens
tctacaactgagcaccactttctgtaactgaacaggcaaagaaattacactgaacatcagcatctggcag

SEQ ID NO: 28 - DNA, 70 bp, Homo sapiens
caggttcaggattaagaaaatggacggaaacatacagctacatacaaatgcaaagcctagtgactaagag

SEQ ID NO: 29 - DNA, 70 bp, Plasmid pUC18
cgaaagggcctcgtgatacgtaggttaatgtcatgataataatggtttcttagacgtcaggtggcacttt

SEQ ID NO: 30 - DNA, 70 bp, Synthetic sequence
tttttttttttttttttttttttttttttttttttttttttttttttttttttttttttttttttttttt

SEQ ID NO: 31 - DNA, 50 bp, M13 lambda phage DNA; identical to SEQ ID NO: 2

SEQ ID NO: 32 - skipped

(19) United States
(12) Patent Application Publication
(10) Pub. No.: US 2008/0050842 A1
(43) Pub. Date: Feb. 28, 2008

(54) METHOD OF VISUALIZATION AND QUANITIFICATION OF BIOPOLYMER MOLECULES IMMOBILIZED ON SOLID SUPPORT

(76) Inventors: Valeri V. Golovlev, Oak Ridge, TN (US); Ye Sun, Knoxville, TN (US); Michael P. McCann, Dudley, NC (US); Wen-Hua Fan, San Diego, CA (US)

Correspondence Address: LUEDEKA, NEELY & GRAHAM, P.C., P O BOX 1871, KNOXVILLE, TN, 37901, US

(21) Appl. No.: 11/551,517

(22) Filed: Oct. 20, 2006

Related U.S. Application Data
(63) Continuation-in-part of application No. 10/776,882, filed on Feb. 11, 2004, now abandoned.
(60) Provisional application No. 60/448,175, filed on Feb. 15, 2003.

(51) Int. Cl. G01N 33/553

(52) U.S. Cl. 436/525; 436/174; 977/904

(57) ABSTRACT

A method and kit are provided for visualization of a latent pattern of molecular structures on a substrate surface. The method is comprised of exposing the substrate to a solution of nano-particles or to a powder of nano-particles. A detectable change is brought about as a result of non-specific binding nano-particles to the chemical groups on the substrate surface carrying the target molecular structures. The invention also provides compositions and kit for practicing the method. Further, the invention provides methods of capturing image of the substrate surface for visualization and quantitation of the molecular structures.

### CLAIM OF PRIORITY

This patent application is Continuation-In-Part of co-pending application Ser. No. 10/776,882, entitled “Method Of Visualization And Quantification Of Biopolymer Molecules Immobilized On Solid Support” filed on Feb. 11, 2004, and also claims the benefit of U.S. Provisional Application No. 60/448,175, entitled “Method Of Visualization And Quantitation Of Biopolymer Molecules Immobilized On Solid Support” filed on Feb. 15, 2003, the specifications of which are incorporated herein by reference in their entireties.

### STATEMENT REGARDING GOVERNMENT RIGHTS IN THIS INVENTION

This invention was made with U.S. government support under Grant Nos. 2R44CA084804 and R43 GM074311 awarded by National Institute of Health. The United States government has certain rights in this invention.

### FIELD

The present invention relates to the field of bio-polymer analysis and detection which is of interest in biomedical research, genetic studies and disease diagnosis, toxicology tests, forensic investigation, agriculture and pharmaceutical development.

### BACKGROUND

Data display technology has become a paramount tool in the information age. The need for data analysts to have large sums and varied types of data at their fingertips has never been more desirable. However, as many types of data analysts, including engineers, soldiers, pilots, and executives have come to realize, the display of too much data at one time has the potential of undermining an original purpose of the data collection—to understand the current status and trends of a particular system or systems. When the addition of data translates into less clarity for an analyst, the perceived benefit of having more information actually becomes a detriment for the analyst. Based on these realities, many data accumulation and display technologies incorporate creative ways to package large sums of data into understandable and meaningful information for an analyst.

For Nucleic acid hybridization has become an increasingly important technology for DNA analysis and gene expression studies. For example, DNA and RNA hybridization techniques are very useful for detecting, identifying, fingerprinting, and mapping molecular structures. Recently developed combinatorial DNA chips, which rely on the specific hybridization of target and probe DNA on a solid surface, attracted tremendous interest from the scientific and medical communities. Although the study of gene activity and molecular mechanisms of disease and drug effects has traditionally focused on genomics, recently proteomics has introduced a very valuable complimentary approach to study the biological functions of a cell. Proteomics involves the qualitative and quantitative measurement of gene activity by detecting and quantifying expressions at the protein level, rather than at the messenger RNA level. Multianalyte assays, also known in the art as “protein chips”, involve the use of multiple antibodies and are directed towards assaying for multiple analytes. The approach enables rapid, simultaneous processing of thousands of proteins employing automation and miniaturization strategy introduced by DNA microarrays.

An attractive feature of microarray technology for genomic applications is that it has the potential to monitor the whole genome on a single chip, so that researchers can have a complete picture of the interaction among thousands of genes simultaneously. Possible applications of DNA microarrays include genetic studies, disease diagnosis, toxicology testing, forensic investigation, and agriculture and pharmaceutical development. Growing applications for microarrays creates new demands for reducing the complexity and improving the detection sensitivity of DNA chips.

Currently, the most common approach to detect DNA bound to a microarray is to label it with a reporter molecule that identifies DNA presence. The reporter molecules emits detectable light when excited by an external light source. Light emitted by a reporter molecule has a characteristic wavelength, which is different from the wavelength of the excitation light, and therefore a detector such as a Charge-Coupled Device (CCD) or a confocal microscope can selectively detect a reporter's emission. Although the use of optical detection methods increases the throughput of the sequencing experiments, the disadvantages are serious. Incorporation of a fluorescent label into a nucleic acid sequence increases the complexity and cost of the entire process. Although the chemistry is commonplace, it necessitates additional steps and reagents for fluorescent labeling, and can be accomplished only with specialized expensive equipment for detection of weak fluorescent signals.

Autoradiography is another common technique for the detection of molecular structures. For DNA sequence analysis applications, oligonucleotide fragments are end labeled, for example, with .sup.32P or .sup.35S. These end labeled fragments are then exposed to X-ray film for a specified amount of time. The amount of film exposure is determined by densitometry and is directly related to the amount of radioactivity of the labeled fragments adjacent to a region of film.

The use of any radioactive label has several disadvantages. First, the use of radioactive isotopes increases the risk of workers acquiring mutation-related diseases. As such, precautions must be implemented when using radioactive markers or labels. Second, the need of an additional processing step and the use of additional chemical reagents and short-lived radioisotopes increases the cost and complexity of this detection technique.

A method of using gold nano-particles as an alternative detection agent for detection of nucleic acids on microarrays without using specialized expensive equipment for detection is taught in U.S. Pat. Nos. 6,495,324 and 6.682,895. The nucleotides having sequence complimentary to the target nucleic acid first are attached to the surface of gold nano-particles (nanoparticle-oligonucleotide conjugates). The gold nano-particles conjugates than hybridized with target molecules hybridized to the probes on microarray surface. In this method the hybridization of gold conjugates marks array spots where target molecules are located. However, the method required a large number of sequence-specific oligonucleotides for manufacturing nano-particle conjugates, which seen as the significant disadvantage of the oligonucleotide-conjugate method. In addition, oligonucleotides-gold conjugates are often unstable under the typical hybridization conditions, which further complicates the use of gold-oligonucleotides conjugates (see Li et al., “Multiple thiol-anchor capped DNA-gold nanoparticle conjugates”, Nuc. Acids Res., 30(7), 1558-1562 (2202)).

Yguerabide et al., U.S. Pat. No. 6,586,193, describes a method of using light scattering for sensitive detection of target biopolymers. In this method another type of metal-conjugate particles described, which conjugates provides specific binding component to bind target molecules through hapten pairs, such as biotin/streptavidin or digoxigenin/antidigoxigenin and the similar binding systems. In some embodiments of the method the particles are coated with, for instance, streptavidin wherein biotin is incorporated into the structure of target molecules during the steps of analyte preparation. Yet, the modification of target molecules by incorporating labeling group(s) (e.g., biotin and the similar) for detection often introduces bias, reduces accuracy and increases the cost and complexity of microarray analysis.

Remacle et al., US App. No. 2003/0096321, describes a method for identification of a labeled target compound on a surface of solid support. In one embodiment of the method the use of non-modified target molecules is described by employing a sandwich type assay, in which the target is hybridized with an additional labeled nucleotide sequence, which labeled nucleotide allows attachment of gold-conjugates to the target compound. Yet, once again, the method requires the use of a large number of labeled sequence-specific oligonucleotides, which makes the method unpractical. The modification of this approach for reducing the number of various labeled sequence-specific oligonucleotides, in which universal binding sequences such as polyT and polyA nucleotides are used, has a limited utility and cannot be used for analysis of partially degraded mRNA, which lost partially or completely the polyA tail or for analysis of microbial mRNA, which do not have polyA tail.

While a large number of detection methods for use with nucleic acids and protein arrays have been described in patents and in the scientific literature, virtually all methods set forth in prior art contain one or more inherent weaknesses. Some lack the sensitivity necessary to accomplish certain tasks. Other methods lack the recognition specificity due to imposing non-optimal conditions for forming probe-target duplexes. Still others are expensive and difficult to implement due to complexity of sample preparation and often have drawbacks due to bias introduced by labeling groups incorporated into the structure of target molecules.

Thus, there is a need for an improved method and kit(s) for visualization of molecular structures, which said method is quantitative, sensitive, and simple to implement. There is also a need for an improved method for visualizing a latent pattern of molecular structures of target molecules on solid support, which method does not required chemical modification/labeling of the target molecules.

Nomenclature

Unless defined otherwise, all technical and scientific terms used above and throughout the text have the same meaning as commonly understood to one of ordinary skill in the art to which this invention belongs.

The following definitions are provided to facilitate a clear understanding of the present invention. The term “molecular structure” refers to a macro-molecule, including organic compound, antibody, antigen, virus particle, metal complex, molecular ion, cellular metabolite, enzyme inhibitor, receptor ligand, nerve agent, peptide, protein, fatty acid, steroid, hormone, narcotic agent, synthetic molecule, medication, nucleic acid single-stranded or double-stranded polymer and equivalents thereof known in the art.

The term “bound molecular structures” or “duplex” refers to a corresponding pair of molecules held together due to mutual affinity or binding capacity, typically specific or non-specific binding or interaction, including biochemical, physiological, and/or pharmaceutical interactions. Herein binding defines a type of interaction that occurs between pairs of molecules including proteins, nucleic acids, glycoproteins, carbohydrates, hormones and the like. Specific examples include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand. etc.

The term “sample substance” refers to a media, often a liquid media, which was prepared for the purpose of analysis and establishing (a) the presence or absence of a particular type of molecular structure; (b) the presence or absence of a plurality of molecular structures; (c) the presence or absence of specific groups of molecular structures; (d) the presence or absence of a specific group on a molecular structure of interest.

The term “target molecular structure” or “target” refers to a molecular structure whose presence or absence in a sample substance needs to be established.

The term “target group” refers to a portion of a molecular structure whose presence or absence in a molecular structure needs to be established.

The term “probe molecular structure” or “probe” refers to a molecular structure of known nature, which said probe is capable of binding to a particular type of target molecular structure or to any agent from a specific class of molecular structures. Said probe is used to witness the presence of the corresponding target molecular structure in a sample substance.

The terms “solid support” and “substrate” are used interchangeably and refer to a structural unit of any size, where said structural unit or substrate is having a flat surface suitable for immobilization of probe molecular structures and said substrate made of a material such as, but not limited to, glass, fused silica, synthetic polymers, and membranes.

The term “nano-particle” or “particle” refers to a particle of any shape having the size in the range of from 0.001 micron to 10 microns and, unless specified otherwise, consisting of any solid material or combination of solid materials or refers to a droplet of liquid phase in a solvent, such as Oil/Water emulsions and the similar.

The term “ionizable chemical group” refers to a portion of a molecule, wherein said molecule is immobilized on surface or is floating free in solution, and where said portion of the molecule is capable of acquiring electric charge due to dissociation in solution or due to forming a complex with electrically charged portion of other molecule(s). Examples of ionizable chemical groups include, but not limited to, the acidic and basic chemical groups dissociating by splitting into a charged molecular fragment and ions of hydrogen or hydroxide respectively. Yet another example of ionizable chemical group is a chemical group capable of forming complex with ions of hydrogen or hydroxide. It is appreciated that a molecule or surface can carry a plurality of ionizable groups of different nature and in many instances the positive and negative ionizable groups can co-exist within the same molecule or positive and negative ionizable groups can be present in close proximity to each other on the surface of substrate or nano-particle.

Throughout the disclosure hereinbelow “interaction of a particle and a substrate” and “binding particle to substrate” means preferably ionic interaction of said particle with all chemical groups present on the substrate surface including the chemical groups of the substrate core material, the chemical groups of layer(s) of materials that can be present on the substrate surface, and chemical groups of probe and target molecular structures that can be bound to the substrate surface, wherein, unless defined otherwise, the plurality of all chemical groups of the substrate core materials, the chemical groups of layer(s) of materials on the substrate surface, the probe and target molecules attached to the substrate surface are referred as “chemical groups on the substrate surface” or “chemical groups of the substrate”.

The term “non-specific binding” refers to interaction of nano-particle and a molecular structure on the surface of substrate which interaction occur most preferably through ionic interaction of the nano-particle and ionizable chemical groups of the substrate and which interaction does not rely on sequence-specific recognition or sequence and structural complimentarity of the molecular structure and nano-particle or nano-particle conjugate. The term “non-specific binding” specifically excludes binding nano-particle conjugate due to hybridization of nucleic acids or binding due to protein-antibody interaction of the nano-particle conjugate and the molecular structure.

It must be noted that as used herein and in the appended claims, the singular forms “a”, “and”, and “the” include plural referents unless the context clearly dictates otherwise. Thus, for example, reference to “a molecular structure” may include a plurality of macro-molecules, including organic compounds, antibodies, antigens, virus particles, metals, metal complexes, ions, cellular metabolites, enzyme inhibitors, receptor ligands, nerve agents, peptides, proteins, fatty acids, steroids, hormones, narcotic agents, synthetic molecules, medications, nucleic acid single-stranded or double-stranded polymers and equivalents thereof known to those skilled in the art, and so forth.

### SUMMARY

The present invention provides an improved method and kit useful for detecting, identifying, fingerprinting, and mapping molecular structures. In accordance with the present invention, the method is capable of simultaneously detecting multiple molecular structures of different type immobilized on solid support in predetermined test sites. In accordance with the present invention nano-particles made of specific materials and carrying a net electric charge are used to visualize and characterize the quantity of target molecular structures on the surface of solid support. The method and kit provided herein substantially eliminates or reduces the disadvantages and problems associated with devices and methods known from prior art.

The method of present invention employs nano-size particles for detection molecular structures of interest, where said nano-size particles are selected from the group of solid particles and particles of liquid phase such as Water/Oil emulsions and the similar. Said solid particles consisting of the group of particles of polymer materials, powders or aqueous suspensions of nano-particles consisting of materials selected from the group of oxides, carbides, nitrides, borides, chalcogenides, metals, alloys, and mixtures thereof. In some embodiments, the solid particles are coated with a substance selected from the group consisting of surfactants, waxes, oils, silyls synthetic and natural polymers, resins, and mixtures thereof. The coatings are selected for their tendency to deliver positive or negative surface electric charge and also their tendency to promote desirable hydrophobic or hydrophilic properties of the particle surface.

It is appreciated that the method of present invention is not bound to any particular assumption or theory of the mechanism of interaction of the chemical groups present on the substrate surface and said nano-particles. The method of present invention can be practiced by many different ways. Various other embodiments and variations to the preferred embodiments will be apparent to those skilled in the art and may be made without departing from the spirit and scope of the following claims.

Now considering specific examples, the preferred solid nano-particles for the method include particles of various metals including gold (Au), silver (Ag), platinum (Pt), aluminum (Al), nickel (Ni), iron (Fe), palladium (Pd), titanium (Ti), scandium (Sc), vanadium (V), chromium (Cr), magnesium (Mg), manganese (Mn), cobalt (Co), copper (Cu), zinc (Zn), yttrium (Y), zirconium (Zr), niobium (Nb), molybdenum (Mo), technetium (Tc), ruthenium (Ru), cadmium (Cd), lutetium (Lu), hafnium (Hf), tungsten (W), rhenium (Re), osmium (Os), iridium (Ir), tantalum (Ta), rhodium (Rh), rare-earth metals ytterbium (Yb), lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutecium (Lu), and alloys thereof. Preferred metal oxides particles include particles of MgO, SrO, BaO, CaO, TiO.sub.2, ZrO.sub.2, FeO, V.sub.2 O.sub.3, V.sub.2 O.sub.5, Mn.sub.2 O.sub.3, Fe.sub.2 O.sub.3, NiO, CuO, Al.sub.2 O.sub.3, SiO.sub.2, ZnO, Ag.sub.2 O, TiSiO.sub.4, ZrSiO.sub.4. rare-earth metal oxides, the corresponding hydroxides of the foregoing, particles and quantum dots of semiconducting materials (Si, CdSe, CdSe/CdS, CdSe/ZnSe, PbS, PbSe, ZnS, GaSb, GaAs, InAs), ceramic nano-particles, and mixtures thereof. Suspensions of nano-particles and nanoscale powders of various compositions can be produced using different methods known in the art. Some illustrative but not exhaustive lists of manufacturing methods include precipitation, hydrothermal processing, combustion, arcing, template synthesis, milling, sputtering and thermal plasma taught by Yadav and Pfaffenbach in US. Pat. App. Nos. 20050274447 and 20050063889, and by Reed et al., in U.S. Pat. No. 6,976,647, each of these patents is herein incorporated by reference in its entirety and specifically for description of various methods of manufacturing nano-particles and methods and instruments for milling and reconstituting powders of nano-particles from solid composites.

Preferred particles of polymer materials include particles of biologically inert latex consisting of carboxylated styrene butadiene, carboxylated polystyrene, carboxylated polystyrene with amino groups, acrylic acid polymers, methacrylic acid polymers, acrylonitrile butadiene styrene, polyvinyl acetate acrylate, polyvinyl pyridine and vinyl-chloride acrylate taught, for instance, by Hager in U.S. Pat. No. 3,857,931, incorporated herein by reference. Particles of polymer material for practicing the method can be manufactured using various manufacturing techniques as reviewed, for instance, by Yeo and Kiran (“Formation of polymer particles with supercritical fluids: A review”, J. of Supercritical Fluids, 34, 287-308 (2005)), incorporated herein by reference in its entirety and specifically for its description of particles of polymer materials and their use in various applications.

It is appreciated that particles composed of core material such as metal, metal oxide, semiconductor, ceramic, or polymer can be encapsulated by a shell of second material, which said second material is essentially different from the material of the particle core. Encapsulation of nano-particles is known in the art for stabilization nano-particles in solutions and has been taught for achieving various desirable properties of nano-particles as described by Fleming and Walt (“Stability and Exchange Studies of Alkanethiol Monolayers on Gold-Nano-particle-Coated Silica Microspheres”, Langmuir, 17(16), 4836-4843 (2001)), and by Eggeman et al (“Synthesis and characterization of silica encapsulated cobalt nano-particles and nano-particle chains”, J. of Magnetism and Magnetic Materials, 301, 336-342 (2006)), incorporated herein by reference.

In the method of present invention the particles have the size in the range of from 0.001 microns to about 10 microns and most preferably have the size in the range of from 0.002 microns to 0.5 microns. The proper selection of the size of the particles is important factor for achieving a desirable binding of nano-particles to the target molecular structures and for reducing undesirable binding of said particles to the probe molecules on the substrate.

In suspension or in powder form, the particles for practicing the method of present invention typically carry surface electric charge in the range of from −1200 mC/m.sup.2 to +1200 mC/m.sup.2 and most preferably carry the surface charge in the rage of from −500 mC/m.sup.2 to +500 mC/m.sup.2. or equally acceptable, have Zeta-potential in the range of from −150 mV to −1 mV or from +1 mV to +150 mV. The various methods suitable for measurements surface charge and Zeta-potential of nano-particles are described, for instance, by Duknin et al., in U.S. Pat. No. 6,915,214, and Aoki in U.S. Pat. No. 6,051,124, each of these patents is herein incorporated by reference in its entirety and specifically for description of methods and instruments for characterization of particle surface charge and particle surface electric properties. In aqueous suspensions said particle surface charge is a function of pH of the solution. In the method of present invention the solution pH is typically selected from the range of from pH=1.0 to pH=11.0, and most preferably from the range of from pH=3.0 to pH=9.0. It is appreciated that in solution the particle may exhibit amphoteric behavior: at high pH the particles may carry negative charge, and at low solution pH the same particles can have positive surface charge. The proper selection of pH and ionic strength of the reaction solution are two important factors for achieving optimal sign and density of the electric charge carried by particles and by substrate on which the latent pattern of molecular structures has to be detected.

For maintaining the desirable surface charge and hydrophobic or hydrophilic property the particles can be modified by deposition of additional layer of coating material. Typical suitable groups of coating materials for maintaining desirable surface charge are those containing an active hydrogen e.g. —COOH, —CONH.sub.2. a nitrile group, a secondary amine group, a primary amine group, trimethylammonium group, or any combination thereof. An additional group of coating materials for controlling hydrophobic or hydrophilic property of particles consists of cationic, anionic, and zwitterionic detergents, bile acid salts, or any combination thereof.

Specific examples of coating materials given by way of illustration and not by way of limitation are: ligands (for instance, thiolates and aminosilanes); Phenylethynyl di-, tri-, and tetrathiols; Alkylthiols and Disulfide-terminated moieties; Tetrapolymers (for instance, N-isopropylacrylamide, oleic and acrylic acid): the Aromatic-oxy-carboxylic acid iron-including compounds; Polyethylene glycol; Ployethylenimine, natural and synthetic polymers with any number of incorporated asparate, asparagine, glutamate, histidine, lysine, or arginine amino acids or any combination thereof; anionic detergents including Chenodeoxycholic acid; Chenodeoxycholic acid sodium salt; Dehydrocholic acid; Deoxycholic acid; Deoxycholic acid: Deoxycholic acid methyl ester; Digitonin; Digitoxigenin; N;N-Dimethyldodecylamine N-oxide; Docusate sodium salt waxy solid; Docusate sodium salt; Glycochenodeoxycholic acid sodium salt; Glycocholic acid hydrate; Glycocholic acid sodium salt hydrate; Glycodeoxycholic acid monohydrate; Glycodeoxycholic acid sodium salt; Glycodeoxycholic acid sodium salt; Glycolithocholic acid 3-sulfate disodium salt; Glycolithocholic acid ethyl ester; N-Lauroylsarcosine sodium salt; N-Lauroylsarcosine sodium salt; N-Lauroylsarcosine solution; N-Lauroylsarcosine solution; Lithium dodecyl sulfate; Lugol solution; Niaproof 4; Niaproof 4; Triton QS-15; Triton QS-44; 1-Octanesulfonic acid sodium salt; 1-Octanesulfonic acid sodium salt; Sodium 1-butanesulfonate; Sodium 1-ecanesulfonate; Sodium 1-decanesulfonate; Sodium 1-dodecanesulfonate; Sodium 1-heptanesulfonate anhydrous, Sodium 1-heptanesulfonate anhydrous; Sodium 1-nonanesulfonate; Sodium 1-propanesulfonate monohydrate; Sodium 2-bromoethanesulfonate; Sodium cholate hydrate; Sodium choleate; Sodium deoxycholate; Sodium deoxycholate monohydrate; Sodium dodecyl sulfate; Sodium hexanesulfonate; Sodium octyl sulfate; Sodium pentanesulfonate; Sodium taurocholate; Taurochenodeoxycholic acid sodium salt; Taurodeoxycholic acid sodium salt monohydrate; Taurohyodeoxycholic acid sodium salt hydrate; Taurolithocholic acid 3-sulfate disodium salt; Tauroursodeoxycholic acid sodium salt; Triton X-200 solution; Triton® XQS-20 solution; Trizma® dodecyl sulfate; Ursodeoxycholic acid; cationic detergents including; Alkyltrimethylammonium bromide; Benzalkonium chloride, Semisolid; Benzalkonium chloride, SigmaUltra; Benzyldimethylhexadecylammonium chloride; Benzyldimethyltetradecylammonium chloride; Benzyldodecyldimethylammonium bromide; Benzyltrimethylammonium tetrachloro iodate; Dimethyldioctadecyl ammonium bromide; Dodecylethyldimethylammonium bromide; Dodecyltrimethylammonium bromide; Dodecyltrimethylammonium bromide; Ethylhexadecyldimethylammonium bromide; Girard's reagent T; Hexadecyltrimethylammonium bromide; Hexadecyltrimethylammonium bromide; N,Nc/,Nc/-Polyoxyethylene(10)-N-tallow-1,3-diaminopropane; Thonzonium bromide; Trimethyl(tetradecyl)ammonium bromide; zwitterionic detergents including; CHAPS; CHAPSO; 3-(Decyldimethylammonio)propanesulfonate inner salt; 3-(Dodecyldimethylammonio)propanesulfonate inner salt; 3-(Dodecyldimethylammonio)propanesulfonate inner salt; 3-(N,N-Dimethylmyristylammonio)propanesulfonate; 3-(N,N-Dimethyloctadecylammonio)propanesulfonate; 3-(N,N-Diminethyloctylammonio)propanesulfonate inner salt; 3-(N,N-Dimethylpalmitylammonio)propanesulfonate; and also detergents cetyltrimethylammonium bromide; bis(2-ethylhexyl)sulfosuccinate sodium salt; decaethylene glycol monododecyl ether; hexaethylene glycol monododecyl ether; polyoxyethylene oleyl ether, Triton X-100; Tween 20; or any combination of various detergents thereof.

Various systems and methods known in the art can be employed for modifying the surface characteristics of nano-particles for practicing method of present invention. The example of methods suitable for modifying surface of nano-particles are disclosed by Yadav, et al in US Pat. Appl. No. 20050084608, by Goldstein in U.S. Pat. No. 7,081,450, and by Wang in U.S. Pat. No. 6,956,084, all incorporated herein by reference in its entirety for their description of methods, processes and instruments for modifying surface of nano-particles.

A further example of treatment for enhancing hydrophobic property of nano-particles is the treatment particles with organic silicon compounds taught by US Pat. App. No. 20050095520, incorporated herein by reference. The hydrophobic-treatment has a procedure of treating with an organic silicon compound and so on that reacts or physically absorbs to the nano-particles in powder form or particles in solution.

Hereinabove, an example of the organic silicon compound is silicone oil. Preferable examples of the silicon oil are dimethylsilicone oil, methylphenylsilicolle oil, alpha-methylstyrene-denatured silicone oil, chlorophenylsilicone oil, fluorine-denatured silicone oil.

The treatment with the silicone oil may have the procedure of direct mixing of the silicone oil and the nano-particle powder treated with the silane coupling agent using a mixer such as a Henschel mixer. It may have the procedure of spraying of the silicone oil to the fine nano-particle powder as a base. It may have the procedures of dissolving or dispersing of the silicone oil to a suitable solvent, mixing the fine silica powder thereto, and then removing the solvent.

Additional examples of the silane coupling agent used for the hydrophobic-treatment and controlling surface charge of nano-particles, are hexamethylenedisilazane, trimethylsilane, trimethylchlorosilane, trimethylethoxysilane, dimethyldichlorosilane, methyltrichlorosilane, allyldimethylchlorosilane, allylphenyldichlorosilane, benzyldimethylchlorosilane, bromomethyidimethylchlorosilane, alpha-chloroethyltrichlorosilane beta-chloroethyltrichlorosilane, chloromethyidimethylchlorosilane, triorganosilylmercaptan, trimethylsilylmercaptan, triorganosilylacrylate, vinyidimethylacetoxysilane, dimethyidiethoxysilane, dimethyldimethoxysilane, diphenyldiethoxysilane, hexamethyldisiloxane, 1,3-divinyltetramethyldisil-oxane, 1,3-diphenyltetramethyidisiloxane, and dimethylpolysiloxane that has 2 to 12 siloxane units per one molecule and has each of the terminal units having a hydroxyl group bound to a silicon atom.

Typical suitable nano-particles for the method are those supplied commercially as powders or as aqueous suspensions or emulsions. Many type of nano-particles are suitable for use in this invention provided they surface charge and hydrophobic or hydrophilic properties are meet the criteria set forth above. This invention comprehends the use of all the suitable particles, including suspensions of solid particles and, equally acceptable, liquid-phase emulsions.

The method of present invention is particularly beneficial for the detection of biopolymer materials immobilized on the surface of solid support, including DNA, RNA, natural and synthetic polynucleotides and polypeptides, proteins and the like known in the art. The method of present invention allows visualizing and quantitatively characterizing probe-target complexes on the substrate surface by exposing and developing the substrate in a solution of nano-particles and, in some embodiments, by exposing the substrate to powder of nano-particles.

Although in order to provide better understanding of the present invention examples of using iron oxide and gold nano-particles for visualization and quantification biopolymers will be presented, it is appreciated that in the method of present invention nano-particles of other materials disclosed hereinabove can be used. It is appreciated that the applications of the teachings of the present invention are in many cases broader than the specific examples or exemplary models. Various other embodiments and variations to the preferred embodiments will is be apparent to those skilled in the art and may be made without departing from the spirit and scope of the method of present invention.

The method of present invention is based on the observation that nano-particles, which meet certain conditions, bind to the surface of a solid support when specific chemical groups are presented on said surface. The binding most preferably occurs due to ionic attraction of nano-particles to the target molecular structures on the surface of solid support.

Now considering gold particles as an example, it is appreciated that the nano-particles of other materials can be used in a similar manner as disclosed herein below. In an aqueous solution, colloidal gold particles normally carry a negative electric charge and show high affinity for positively charged chemical groups, though negatively charged chemical groups on the surface repel the particles. To maximize the affinity of nano-particles to a specific surface or chemical groups, said nano-particles could be coated with different modifiers, such as surfactants, waxes, oils, silyls, synthetic and natural polymers, resins, and mixtures thereof. When attached to the particle, the modifier(s) adjust particle net electric charge and particle's hydrophobic or hydrophilic properties and, in this fashion contributes to higher affinity of the particle to the negatively or positively charged chemical groups of the probe and target molecules on the substrate surface.

The nano-particles bind to the surface and cover it with a layer, where the density of the particles in said layer represents the density of attracting or repelling chemical groups on the surface. Indeed, for colloidal particle having the size of 1 nm or larger, the net force that attracts or repels the particle to or from the surface represents the average force from all chemical groups and electric charges on the surface in the area covered by particle “footprint”, which “footprint” is determined by particle size and the screening length of electric charge in solution. Said all chemical groups and electric charges on the substrate surface include the chemical groups of the substrate core material, the chemical groups of layer(s) of materials that can be present on the substrate surface, and the probe and target molecular structures that can be bound to the substrate surface inside area covered by particle footprint. The nano-particles are bind to the surface when the attraction forces dominate and repel from the surface when the repulsion forces prevail. The density of nano-particles on the surface is related to the net number of attractive and repulsive groups of the substrate core material, the material of coating layer(s) which can be present on the substrate surface, and ionizable groups of probe and target molecules at a corresponding location on the surface. This particular mechanism of binding nano-particles to the surface is presented herein in order to provide a better understanding of the present invention and is given by way of illustration, and not be a way of limitation.

It is appreciated that the method is not bound to any particular assumption or theory of the mechanism of interaction of the surface and the colloidal particle, and said method can be practiced by many different ways. Various other embodiments and variations to the preferred embodiments will be apparent to those skilled in the art and may be made without departing from the spirit and scope of the method disclosed herein.

The method of present invention is different from the conventional detection techniques known in the art, in which techniques labels bind to the targets by a specific, one-to-one interaction of homologous hybridized sequences or due to sequence and structural complimentarity of molecular fragments of the probe and target molecules, such as antibody-antigen interaction. Indeed, in the method of present invention the binding is driven by a non-specific, one-to-many ionic interaction, in which the binding force applied to a single particle represents an average of many attracting and repulsing forces from many chemical groups of the probe and target molecule and the substrate surface.

A new and unexpected result of the present invention vs. the methods known in the art is that non-specific ionic binding of the nano-particles to the substrate surface carrying molecular structures provides a sensitive and convenient approach for visualization and quantification of molecular structures on the surface.

The additional new and unexpected result of the present invention is that a broader group of materials and reagents can be used to manufacture nano-particles for detection various biological targets on solid substrates.

Yet, further new and unexpected result of the present invention is that non-specific binding of the particles to the site of the interest on the surface can be accomplished without chemical modification of target molecules, thus eliminating an element of previous art.

Yet, another new and unexpected result of the present invention is that by selecting the size and chemical composition of nano-particles, pH and ionic strength of the reaction solution the conditions can be set for selective binding of the particles to the target molecules without undesirable binding said particles to the probe molecules on the substrate.

An overall advantage of the method of present invention is seen as providing means for overcoming the drawbacks of the methods known in the art by introducing a new method for visualizing biopolymer molecules immobilized on a surface. This new method is quantitative, more sensitive, does not required chemical modification of target molecules for detection, does not interfere with the probe-target binding or hybridization, can be implemented with a large variety of probe and target molecular structures, and can be carried out using inexpensive detection equipment including, but not limited to, an optical scanner, an optical microscope equipped with a camera, and various photoequipment for capturing still and video images, by detecting local magnetic and electroconductive properties of the substrate with the particles attached, or by using magnetic resonance spectroscopy.

Another aspect of the invention is a kit for the practice of the method. The kit comprises multiple containers having appropriate amounts of reagents necessary to practice the method, including some or all of the following: a container containing a suitable colloidal solution or powder; a container containing an activating solution; a container containing a buffer solution for preparing colloidal solution at desirable pH and ionic strength; a container containing capping solution for blocking the substrate prior to development in a colloidal solution; a container or attachable chamber suitable to carry out hybridization or a binding reaction; and a container suitable for washing the substrate by dipping in or rinsing with a washing buffer. Additionally, the kit can include a set of substrates suitable for immobilization of probe molecular structures, i.e., producing microarrays, and a cassette for capturing diffuse reflectance from a transparent substrate when using an optical scanner or camera.

For quantification of the hybridized target molecules, surface of the solid support (i.e., microarray) covered by nano-particles can be analyzed and density of the bound nano-particles can be measured using conventional optical techniques and a suitable image-capturing apparatus. Here, the suitable image-capturing apparatus can include any device of plurality of devices capable of acquiring absorbance on the surface and reflectance from the surface of interest, and most preferably, includes flatbed scanners. The resolution of the image-capturing device must be sufficient to identify optical response from individual test sites on the surface of the substrate. Most preferably, the image-capturing device must be able to digitize the captured image and transfer the image to a computer for storage and further analysis. It is appreciated that image of the same area of the substrate can be captured multiple times for averaging, reducing noise, color manipulations, filtering and performing other image-processing operations known to one skilled in the art. Specialized software can be implemented for obtaining quantitative characteristics of the optical response from each individual test site on the substrate. These quantitative parameters can be used to quantify the distribution of nano-particles bound to the substrate and accordingly to measure the quantity of molecular structure of interest in corresponded site(s) of the substrate.

### BRIEF DESCRIPTION OF THE DRAWINGS

FIG. 1 In order to more fully understand the manner in which the above-recited advantages and other objectives of the invention are obtained, a more particular description of the invention described above will be illustrated in the appended drawings. Understanding that these drawings depict only typical embodiments of the invention and are not to be considered limiting of its scope, the invention is further explained and illustrated with additional specificity and detail through the use of the accompanying drawings in which:

FIG. 1: schematic of diffusely and specular reflected light from a substrate illuminated by an external light source, where 1-1 is the substrate, 1-2 is the nano-particles bound to the substrate surface.

FIG. 2: schematic of detection of the specular reflected light from the substrate surface; where 2-1 is the substrate, 2-2 is the nano-particles 2-3 is the light absorbing paint on the back of the substrate; the sensor of image capturing device is placed in direction in which specular reflected light is propagated.

FIG. 3: schematic of detection of the diffusely reflected light from the substrate surface; here the sensor of image capturing device is placed in a direction in which only diffuse reflected light is propagated (e.g., dark-field detection mode); where 3-1 is the substrate, 3-2 is the nano-particles 3-3 is the light absorbing paint on the back of the substrate.

FIG. 4 is schematic of detection of the diffusely reflected light from a transparent substrate surface; here 4-1 is the substrate. 4-2 is the nano-particles and 4-3 is the light absorbing screen placed behind the substrate which screen is absorbing and reducing intensity of light components other than scattered reflection from the front surface of the substrate; the light absorbing screen normally placed behind the substrate on a distance exceeding the focal depth of an image capturing device used to acquire image of the substrate surface.

FIG. 5: schematic of nano-particle placed in close proximity to the substrate surface; the size of the particle, r, and the distance on which an electric charge in solution is screened by solution's free ions, λ.sub.D, define particle's “footprint”, e.g., an area on the substrate which provides the main contribution to the binding between the substrate and nano-particle, wherein in some embodiments of the method the size of particle's footprint, r′, is given by r′=(2 r λ.sub.D−r.sup.2).sup.(½) for λ.sub.D<r and r′=r for λ.sub.D>r.

FIG. 6: schematic of nano-particles placed in a close proximity to the substrate where (I) illustrates the substrate area with no probe or target molecules on the surface; (II) illustrates the substrate area carrying the probe molecule; (III) illustrates the substrate area carrying probe-target duplex on the substrate surface.

FIG. 7: given by way of illustration and not by way of limitation (A) shows the plot of the substrate's surface electric charge inside particle footprint vs. the solution pH for corresponding substrate areas from FIG. 6, where plot (A-I) is for area of silicon oxide substrate carrying positive amino-groups on the surface at the density of 8 groups/nm.sup.2; plot (A-II) is for the substrate area carrying a probe molecule Such as 50-nt long oligonucleotide; plot (A-III) is for the substrate area carrying a probe-target duplex such as 50-nt oligonucleotide probe and 1200-nt RNA molecule; and plot (A-IV) is for surface charge of gold nano-particle carrying positive amino-groups on its surface at density of 0.08 groups/nm.sup.2; the plot (B) shows the plot of the energy of interaction, E, of particle and substrate vs. the solution pH for the substrate area carrying (E-II) probe molecule inside particle footprint; (E-III) for a substrate area carrying probe and target molecules inside the particle's footprint; here in E-II and E-III E>0 indicates repulsion between the particle and substrate and E<0 indicates attraction between the particle and the substrate.

FIG. 8: shown are three images (A-C) of the substrate carrying total four spots of M13 phage single-stranded DNA (7,200-nt ssDNA) at surface density of 10, 3.3. 1.1, and 0.34 ng/mm.sup.2 and carrying total four spots of 60-nt long oligonucleotide with the sequence of (5′-ccaggtcaccttgggctctgtttgtcagatcctgttatccatagcctttagagaggacct-3′) tethered on the substrate surface at density of 10, 3.3, 1.1, and 0.34 ng/mm.sup.2; the substrate was developed in solution of 250-nm cationic gold particles at solution pH of pH =3.0 (image A), pH=4.0 (image B), and pH =7.0 (image C); the solution with pH=4.0 allows selective detection of the 7.200-nt target DNA, with about no signal detected from the spots carrying 50-nt nucleotide probes.

FIG. 9: shown is the image of microarray surface with pattern of hybridized synthetic oligonucleotides visualized by developing the substrate in solution of 8-nm iron oxide particles in 5 mM Tris-HCl buffer; here detection of targets is achieved without any modification/labeling of target molecules and without using sequence-specific agents or antibodies attached to nano-particles as commonly taught in the art for discriminating targets by sequence or by employing antibody-antigen interaction for recognition of target molecules.

FIG. 10: shown is a set up for exposing substrate to a powder of nano-particles; wherein 10-1 is a chamber partially filled with the powder of nano-particles, 10-2 is the substrate carrying the latent pattern of molecular structures on its surface, and 10-3 is the powder of nano-particles.

FIG. 11: (A) shows image of the substrate with pattern of oligonucleotide spots visualized by exposing the substrate surface to powder of 8-nm colloidal particle; drawing (B) shows the layout of oligonucleotide spots on the substrate (A) with the concentration of spotting solutions of two oligonucleotides, Oligo 1 and Oligo 2, for each corresponding spot on the substrate; here the detection of targets is achieved without any modification/labeling of target molecules and without using sequence-specific agents or antibodies attached to nano-particles for discriminating targets by sequence or by employing antibody-antigen interaction for recognition of target molecules.

FIG. 12: plot of intensity of corresponding spots detected on the microarray image in FIG. 11(A) vs. the concentration of spotting solution.

FIG. 13: shown is the image of microarray surface with the pattern of hybridized synthetic oligonucleotides visualized by exposing the substrate to the powder of 8-nm iron oxide particles; here no modification or coating of nano-particles with nucleic acids, proteins, antibodies or any other biopolymer agents has been used to facilitate binding of nano-particles to the target molecules on the microarray.

FIG. 14: shown is image of pattern of poly-L-lysine molecules immobilized on a substrate and visualized by developing the substrate in solution of 250 nm gold particles; the density of the immobilized poly-L-lysine decreased from top to bottom and from left to right and is (a) 1 ng/.mu.l, (b) 0.8 ng/.mu.l, (c) 0.6 ng/.mu.l, (d) 0.5 ng/.mu.l, (e) 0.4 ng/.mu.l, (f) 0.3 ng/.mu.l, (g) 0.2 ng/.mu.l, (h) 0.1 ng/.mu.l, and (i) 0.05 ng/.mu.l.

FIG. 15: Amplitude of the diffusely reflected light at the center of a spot as shown in FIG. 14 vs. the amount of poly-L-lysine in corresponding spot of the image in FIG. 14.

FIG. 16: shown are two images of the substrate with M13 phage DNA immobilized on the Mylar substrate activated with 0.1% solution of .gamma-aminopropyltriethoxylsilane; the latent pattern of DNA spots on the substrate surface was produced by pipetting 1 .mu.l of solutions contained 1 ng/.mu.l (column #1), 10 ng/.mu.l (column #2), and 100 ng/.mu.L (column #3) of DNA; in image (A) the latent pattern was visualized by developing the substrate in solution of 250 nm non-modified, i.e., negatively charged gold particles for 15 min at room temperature; in image (B) the pattern of molecular structures on the substrate surface was visualized by developing the substrate in solution of 250-nm cationic gold particles; the cationic nano-particles have been prepared by adding 80 .mu.l of 0.01% poly-L-lysine (Sigma-Aldrich, Cat. No. P8920) to 1 ml of gold colloid at concentration 3.6.times.10.sup.8 particles/ml in aqueous solution; the mixture was incubated at room temperature at constant shaking for 2 hours; the solution was centrifuged to precipitate nano-particles and the natant carrying the residual unbound poly-L-lysine was discarded; the pellet of nano-particles was resuspended in distilled deionized water to the original concentration of 3.6.times.10.sup.8 particles/ml and this colloidal solution was used to develop the latent pattern of the DNA molecules on the substrate by dipping the substrate into the colloidal solution for 15 min at room temperature; when developing was completed, the substrate was washed gently using distilled deionized water, dried by centrifugation and scanned using Epson Perfection 3200 flatbed scanner.

FIG. 17: shown is the image of microarray surface with pattern of hybridized synthetic oligonucleotides visualized by developing the substrate in solution of 250-nm gold particles in 5 mM potassium biphthalate buffer at pH=4.0; here the detection of targets is achieved without any modification/labeling of target molecules and without using sequence-specific agents or antibodies attached to nano-particles for recognition of target molecules.

FIG. 18: detection of protein A (spots #1) and ImG (spots #2) immobilized on amino-modified Mylar substrate, activated by treatment in 0.1% solution of gamma. aminopropyltriethoxylsilane; the substrate was developed by dipping for 5 min into a solution of 250-nm negatively-charged gold particles (Cat. No. EMGC250, BBInternational, UK) at a concentration 1.times.10.sup.8 particles/ml; spots #3 are negative (e.g., no probe or target) control and spots #4 carry immobilized protein A, which spots have been exposed to solution of ImG.

FIG. 19: A and B are images of microarray substrate with mRNA form Jurkat cells hybridized to synthetic oligonucleotide probes; image A is for RNA sample from normal cell and image B is for RNA sample from iomicine stimulated cells; the pattern of hybridized RNA was visualized by developing substrate in solution of 250-nm cationic gold particles (Cat. No. AG14, Sci-Tec, Inc., Knoxville, Tenn.) in 5 mM potassium biphthalate buffer at pH=4.0; here no modification or coating of nano-particles with nucleic acids, proteins, antibodies or any other biopolymer agents for achieving specific recognition and binding of nano-particles to the target molecules.

FIG. 20: shown is the pattern of differential expression of genes, where the pattern is produced by computer processing of two images of gene expression in FIG. 19, and wherein corresponding images of molecular structures on the substrate in FIG. 19 have been developed according to the method of present invention.

### DETAILED DESCRIPTION

The following describes steps involved in the detection method of present invention, materials, amounts of reagents and other variables such as time and temperature of the steps. The following also describes how a quantitative measurement of the number of biopolymer molecules of interest can be carried out. In order to provide a better understanding of the present invention specific examples are given by way of illustration and not by way of limitation.

For the purpose of immobilization of probe biopolymer molecules on the surface of a solid support, said surface of solid support can be activated using techniques of surface activation known in the art including, but not limited to, activation using amine reactive chemistries, sulfhydryl reactive chemistries, carbonyl reactive chemistries, hydroxyl reactive chemistries, active hydrogen reactive chemistries, silanation chemistries, and the like, see G. T. Hermanson, et al, “Immobilized Affinity Ligand Techniques”, Academic Press (1992), all activation techniques are included herein by reference. Equally acceptable, surface preparation for immobilization of probe biopolymer may include treating and coating the surface by mediating binding agents such as poly-L-lysine, poly-l-glutamic acid, poly-l-aspartic acid. glycine, alanine, cysteine and the like.

Once activated, the surface of solid support can be used for immobilization of probe biopolymer molecules by following known techniques and protocols for immobilization of nucleic acids. DNA. RNA. and proteins, which also include antibodies and antigens and the like, see Hegde, P. et al. “A Concise Guide to cDNA Micro-Array Analysis”, BioTechniques 29, 549-562 (2000); Rehman, et al, “Immobilization of Acrylamide-modified oligonucleotides by copolymerization”. Nucleic Acids Res., v. 27, p.649-655; Eisen. et al, “DNA Arrays of Gene Expression”, Methods Enzymol., v. 303. p. 179-205, all immobilization techniques and protocols are incorporated herein by reference. Immobilization of probe biopolymers results in allocation of known types of probe agents at known locations on the surface. Also true is that the specific location on the surface can be used to identify the type of probe biopolymer molecules at that specific location. An acceptable density of immobilized probe molecular structures ranges from about 0.01 ng/mm.sup.2 to 50 ng/mm.sup.2. preferably from about 0.05 ng/mm.sup.2 to about 10 ng/mm.sup.2 and more preferably from about 0.1 ng/mm.sup.2 to about 1 ng/mm.sup.2.

When immobilization of the probe molecules on the surface has been completed, an additional step of blocking the surface of the solid support can be performed. Blocking prevents non-specific binding of target molecules to the solid support. The blocking also can be used to allocate specific chemical groups on the surface for maintaining desirable positive or negative net surface charge on the substrate surface. Different reagents can be used to block or cap an activated solid support, whereby blocking agents couple and block residual active sites and essentially eliminate said sites from non-specific binding of target biopolymers. Common blocking or capping agents can include glycine, ethanolamine, tris(hydroxymethyl)aminomethane, mercaptoethanol, mercaptoethylamine, cysteine, acetic anhydryde, succinic anhydride, albumine, sodium borohidrade, ammonium chloride, sodium acrylate, etc. Maintaining desirable electric charge on the surface can be achieved by using poly-L-lysine, anionic and cathionic polymers, for instance, PDDA, amino- and mercapto-silane derivatives, etc. One skilled in the art will adjust concentration and time to optimize blocking treatment to a specific type of chemistry used to activate the solid support.

The binding or hybridization operation is performed during which the solid support with immobilized probe biopolymers is exposed to a solution of target molecules. Target molecules bind to the homologous probes on the surface of the solid support. Specificity of the binding can be enhanced by optimizing pH, ionic strength, and temperature of the buffer solution in which binding/hybridization is performed. Duration of the binding operation is another important parameter, which can be used for maximizing specificity of binding process.

The binding operation usually is completed when probe or analyte molecules available for binding are exhausted. However, in some embodiments of this invention the binding operation can be terminated after a predefined reaction time by replacing the hybridization solution with a solution, which is free of analyte/target molecules.

When binding is complete, an optional additional step of modification of the surface of solid support can be implemented by exposing the surface to reagents such as small organic molecules, polynucleotides, peptides and proteins, thus causing these reagents to be immobilized on the surface. This optional step modifies the affinity of the surface to the nano-particles, which improves the visualization and measurements of molecular structures bound to the surface.

Yet, in another embodiment of the present invention after completion of binding of probe and target molecules the substrate is exposed to a solution containing one or more enzymes. which enzymes are capable to digest unbound molecular structures on the substrate surface. Examples of such enzymes includes S1 nuclease, Mung Beam Nuclease, and Exonuclease I, which provided herein by way of illustration and not by way of limitation. Now considering S1 nuclease as an example, the S1 nuclease is isolated from Aspergillus oryzae and is available from various vendors (see, for instance, Startagene, Promega, etc.). S1 degrades single-stranded nucleic acids, although double-stranded RNA, DNA and RNA-DNA hybrids are resistant to S1 nuclease digestion unless large excess of enzyme is used. To achieve satisfactory results one skilled in the art will adjust concentration of enzyme solution, temperature and time of treatment to obtain desirable removal of single-stranded probe molecules. In this embodiment of the present invention the enzymatic digestion of unbound probe molecules provides better discrimination between bound and unbound molecular structures when both probe and probe-target complexes can initiate a detectable precipitation of colloidal particle. This embodiment of the present invention is especially beneficial for identification of a presence of specific molecular structures in a sample substance, although it also can be used to identify an absence of specific molecular structures in a sample substance.

Alternatively, in yet another embodiment of the present invention after completion of binding of the targets and probes immobilized on the substrate the substrate surface is exposed to a solution containing one or more enzymes, which enzymes are capable to digest preferably bound probe-target molecular structures on the substrate surface. One example of such enzyme is Exonuclease III (from E. coli), which provided herein by way of illustration and not by way of limitation. Exonuclease III digest double-stranded DNA and can be used for enzymatic digestion of bound probe molecules. This enzymatic treatment allows identification of sites where no binding reaction occurs most preferably due to the absence of corresponded target molecular structures in a sample substance. Therefore, this embodiment of the present invention is most preferable for identification of absence of specific molecular structures in a sample substance, although it also can be used to identify a presence of specific molecular structures in a sample substance.

During the binding and post binding treatment disclosed herein above a latent pattern of molecular structures is formed on the substrate surface. This pattern now can be visualized by exposing, i.e., developing the substrate in a solution of nano-particles or by applying a powder of nano-particles to the substrate area where the latent pattern of molecular structures is located. During the development step particles are bind to the substrate surface and molecular structures on said surface thereby producing a thin layer of colloidal material on the surface. The density of colloidal material varies from site to site following the pattern of molecular structures on the surface. Therefore. by measuring the density of colloidal material on the surface it is possible to identify the location and also it is possible to measure the quantity of probe-target complexes on corresponding sites of the surface. Here, when solution or suspensions of nano-particles is used for visualization of molecular structures, the concentration of nano-particles in solution and the temperature influences the rate of development of the image. While solutions that are used may be at a starting temperature of about 0.degree. C. or even below, the development temperature is generally maintained in the range of about 1.degree. C. to about 90.degree. C. The results from 4.degree. C. to 50.degree. C. depending on the nature of the sample, appears preferable. Temperatures below 20.degree. C. can also be used to prevent denaturation of probe-target complexes providing latent pattern development is controlled. The temperature, if not controlled during the development, may rise above the preferred ranges. Temperature requirements may be varied by one skilled in the art depending on the nature, characteristics, and the chemical components of the developing solution.

Yet, in another embodiment of the present invention, the solid support with latent pattern of probe-target complexes is exposed to a solution. containing a mixture of nano-particles and an alternative binding agent. Said binding agent is repelled, i.e., not bound to the nano-particles. Different reagents may be used as an alternative binding agent, including small organic molecules, biopolymers, including DNA, RNA, peptides proteins, and the like. One particular example of such an alternative binding agent for use with gold colloids, which is given herein by way of illustration and not be way of limitation, is albumin molecules, and more specifically bovine serum albumin (BSA). In this embodiment of the invention, when the alternative binding agent binds to the surface of the solid support, it blocks the surface and prevent nano-particles from binding to the same spot on the surface. The binding of the nano-particles and alternative binding agent continue until the equilibrium is reached or until reagents are exhausted. In such an arrangement the density of colloidal material on the substrate surface represents the difference of the binding rate of the nano-particles and the binding agent. Said difference of the binding rates usually is varied from site-to-site throughout the surface due to presence or absence of the probe-target complexes in the corresponded sites of the surface. Therefore, by measuring the density of colloidal material on the surface it is possible to identify location and measure the quantity of probe-target complexes on the surface.

The development step is carried out for a period adequate to develop the latent pattern satisfactorily. Usually about 2 to about 60 minutes, or preferably about 5 to about 30 minutes, will be sufficient. For optimal image development, one skilled in the art may vary the concentration of nano-particles, the alternative binding agent, if such is present in solution, the temperature and the treatment time.

Yet, in another embodiment of the present invention the development solution is prepared by mixing a solution of nano-particles and binding agent such as small organic molecules, biopolymers, peptides, proteins, and the like, in which the binding agent is capable binding to the substrate as well as to nano-particles. One particular example of such binding agent, which is given herein by way of illustration and not be way of limitation, is poly-L-lysine molecules. In this embodiment of the invention, the rate of binding nano-particles to the surface is given by the difference in the rate of binding of the agent to the nano-particles and the rate of binding of the agent to the sites of the substrate. Said difference of the binding rates usually is varied from site-to-site throughout the surface due to presence or absence of the probe-target complexes in the corresponded sites of the surface. Therefore, by measuring the density of nano-particles bound on the surface it is possible to identify the location and measure the quantity of probe-target complexes on the surface. A new and unexpected result of the present embodiment of the invention is that the developing process is self-regulated. The development reaction is self-terminating and precipitation of the colloid stops when the binding agent saturates the nano-particles and the substrate. This can be used to prevent the substrate from overdeveloping when exposing it to the developing solution for a substantially longer tine than normal.

After development is complete, non-bound nano-particles are removed by washing the substrate in an appropriate solvent or buffer solution, and most preferably in distilled deionized water.

Yet, in another embodiment of the method the substrate carrying latent pattern of molecular structures is exposed to the powder of nano-particles. Different techniques of exposing the substrate to the particles can be used according to the method of present invention. In one example a chamber can be attached to the substrate. According to the method, the chamber represents any type of container having any size and shape, provided the container creates a confined space around the substrate surface carrying the latent pattern of molecular structures.

The chamber is partially filled with a powder of nano-particles. It is appreciated that the exact amount of the powder of nano-particles loaded to the chamber can varied according to the specific requirements of each application and can be in the range of from 0.01% to about 100% of the volume of the chamber. In one embodiment, the substrate with the chamber, which is partially filled with the powder of nano-particles, is placed in a device for shaking the substrate with the chamber attached. The suitable device to use is a shaker device capable of moving or shaking the substrate with the chamber attached in the plane of the substrate surface. The moving and shaking the substrate causes particles in the chamber move in a regular, semiregular or random pattern all over the substrate surface where the latent pattern of molecular structures has to be detected. The movement of the nano-particles allows each and every spot of the latent pattern on the substrate to be covered by nano-particles at least for a short period of time.

Yet, in another embodiment of the method, nano-particles of magnetic materials can be moved by magnetic force. According to the method, the substrate with the attached chamber can be placed on top of a magnetic stirrer. Most of the known in the art magnetic stirrers for mixing chemical reagents can be used. The magnetic stirrer cause the magnetic nano-particles move over the substrate surface in a regular, semiregular or random pattern all over the substrate surface and allows each and every spot of the latent pattern to be covered by nano-particles at least for a short period of time.

Yet, in another embodiment, the nano-particles can be moved by electrostatic force by exposing the substrate with the chamber attached to alternative or continuous electric filed. The magnitude and geometry of the electric field have to be configured to allow particles movement over the substrate surface in a regular, semiregular or random pattern all over the substrate surface and allows each and every spot of the latent pattern on the substrate to be covered by nano-particles at least for a short period of time.

Yet in another embodiment of the present invention, the substrate with the latent pattern of molecular structures can be exposed to airflow carrying powder of the nano-particles. The airflow carrying nano-particles allows each and every spot of the substrate to be exposed to the nano-particles at least for a short period of time.

It is appreciated that the powder of nano-particles can be prepared by mixing nano-particles having different chemical composition and properties for achieving the optimal treatment conditions. One particular example of preparing a mixture of particles would be preparation of a mixture of magnetic nano-particles and one or mixture of the following: MgO, SrO, BaO, CaO, TiO.sub.2, ZrO.sub.2, FeO, V.sub.2 O.sub.3, V.sub.2 O.sub.5, Mn.sub.2 O.sub.3, NiO, CuO. Al.sub.2 O.sub.3, SiO.sub.2, ZnO, Ag.sub.2 0, TiSiO.sub.4, ZrSiO.sub.4, rare-earth metal oxides, the corresponding hydroxides of the foregoing, particles and quantum dots of semiconducting materials (Si, CdSe, CdSe/CdS, CdSe/ZnSe, PbS, PbSe, ZnS, GaSb, GaAs, InAs), and ceramic nano-particles. The mixture can be moved and controlled by applying a magnetic force while the binding of the particles to the molecular structures on the substrate is controlled by property of various particles in the mixture.

The substrate surface is typically treated with a powder of nano-particles for a period of time from a few seconds to a few hours and more preferably from 1 sec to less than 1 hour. Consequently the particles and the chamber are disposed and the substrate surface is cleaned, for instance, by a flow of compressed air, or by applying a magnetic or electric field or by using any other appropriate techniques for cleaning the substrate surface from the excess of the particles. However, after cleaning the surface some particles are remaining on the surface due to close-range molecular attraction of the nano-particles and molecular structures present on the substrate surface. The distribution of the density of particles on the substrate follows the distribution of latent pattern of the molecular structures. The image of the pattern can be captured by techniques known in the art including optical techniques for measuring absorbance and scattering of the particles, or by measuring local magnetic and electroconductive properties of the surface for detecting magnetic and metal particles, or by employing methods of magnetic resonance spectroscopy for capturing the pattern of magnetically responsive particles.

The image of the substrate surface with nano-particles bound to the surface is captured using conventional methods and equipment for capturing optical images such as a photocamera, an optical microscope equipped with a camera, or by using an optical scanner. For optimal image appearance, one skilled in the art may arrange different ways of illuminating substrate such that (a) the image is created due to light absorbing property of the nano-particles; (b) the image is created by light specular reflected by substrate surface carrying bound nano-particles: and (c) the image is created by light diffusely reflected by substrate surface carrying bound nano-particles., see for example, Golovlev, et al, “Digital Imaging for Documenting and Modeling the Visual Appearance of 19th Century Daguerreotypes”, The J. Imaging Sci. and Technology. vol. 46, 1-7 (2002). It is contemplated that capturing the image created by specular reflectance will be the most beneficial when the size of individual colloidal particle is about 50 nm or smaller. It is considered to be more advantageous to capture image created by diffuse reflectance from the substrate surface when the size of the individual colloidal particle is about 50 nm or bigger. To capture mostly the diffuse reflectance from the substrate surface an opaque substrate can be employed, or equally acceptable, the back-side of the transparent substrate can be painted with a light absorbing paint or, equally acceptable, light absorbing screen can be placed behind the transparent substrate by employing an appropriately designed slide-carrying cassette. Said cassette comprising the light absorbing screen and means for maintaining the distance between the screen and the substrate surface. Here, the distance between the screen and the substrate surface must be bigger than a focal depth of the device employed to capture the image. The distance is usually not less than 1 mm and preferably more than 1 mm, and more preferably from 5 mm to 100 mm.

The method of current invention can be practiced using different types of substrates including glass, fused silica substrates, and substrates made of synthetic polymer materials, for instance polyethylene and its derivatives, polyethylene terephthalate (PET) and its derivatives, polyacrylamide and its derivatives, polymethacrylate and its derivatives. polysterene/divinylbenzene and its derivatives, and the like known in the art. One particular example of the synthetic polymer substrate, which is given here by way of illustration, and not be way of limitation is Mylar.sup.™ polymer films. The Mylar.sup.™ polymer film has appealing surface properties. The polymeric surface is hydrophobic, which allows better control over the shape and size of printed microarray spots. At neutral pH the surface is negatively charged and when exposed to a solution of colloidal gold it repels negatively charged gold particles. However, the surface can be modified and acquires positively charged when treated in solution of a.gamma.-aminopropyltriethoxylsilane or is exposed to a solution of poly-L-lysine. This modified Mylar.sup.™0 film bind negatively charged gold particles. When gold particles precipitate on the surface, the density of the particles can be quantitatively characterized by measuring the diffuse reflectance of the surface.

### EXAMPLE 1

To ease understanding of the concepts taught herein, the following example is presented for describing interaction of nano-particles and a solid substrate with latent pattern of molecular structures on the substrate surface. However, the applications of the teachings of the present invention are in many cases broader than the specific examples or exemplary models and do not depend of any specific assumption of mechanisms of interaction of nano-particles and molecular structures on the substrate surface. Accordingly, the basic teachings are readily modified and adapted to encompass various other embodiments of the method of present invention.

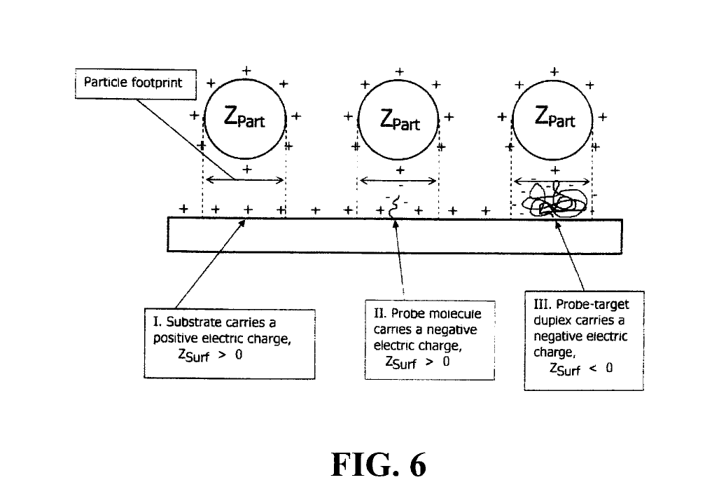

In the following exemplary model, the interaction between electrically charged colloidal particle and microarray surface is driven by two main parameters: the net electric charge of the particle, Z.sub.Part, and the local density of electric charge, z.sub.Surf, on microarray surface in the area covered by particle's footprint as illustrated in FIG. 6. The electric charge on the surface, either the particle surface or substrate surface, is given by the sum of electric charges of all ionized chemical groups carried by the respective surface and by molecular structures, which structures can be present on the surface. With respect to microarrays manufactured on amino-silated glass substrate with nucleic acid probe and target molecules tethered on the array surface, the surface charge is mostly determined by density of SiO.sup.minus groups of glass substrate, the density of positively-charged amino-silane ions R-NH.sub.3.sup.plus on the substrate surface, and the density of negatively-charged phosphate groups PO.sub.4.sup.minus of nucleic acid backbone of the probe and target molecules tethered on the substrate.

The energy, E, of interaction of the nano-particle with the substrate surface is proportional to the multiplication of the electric charge of the particle and the substrate: E˜(Z.sub.Part×z.sub.Surf). The energy E is positive when the particle and the substrate carry the same sign electric charge. Given that the same sign electric charges repel each other, at E>0 the particle is repelled from the substrate. Yet in the other instance when the particle and the substrate carrying opposite sign electric charge the binding energy is negative: E<0. Due to attraction of opposite electric charge the negative energy E herein represents the case of mutual attraction/binding of the particle and the substrate.

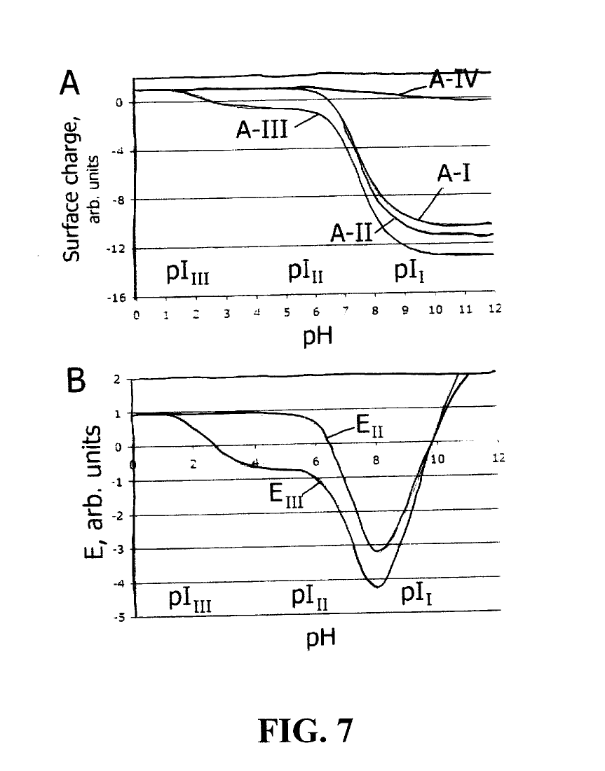

It is appreciated that in aqueous solutions the surface charge of the particle and the substrate are functions of solution pH. For purpose of illustration, and not for purpose of limitation, the dependences of surface charge vs. solution pH known in the art as titration curves are shown in FIG. 7A, where (A-I) is the plot for the substrate area with no probe and target molecules on the substrate; (A-II) is the plot for the substrate area carrying probe oligonucleotides and no target molecules; (A-III) is the plot for the substrate area carrying probe and target oligonucleotides; and (A-IV) is the plot for a nano-particle carrying positive ionized groups on the particle's surface. The pH of solution at which the surface charge is zero is referred as the surface isoelectric point pI. For three substrate areas I-III in FIG. 7A: pI.sub.III<pI.sub.II<pI.sub.I, where pI.sub.I, pI.sub.II, and pI.sub.III, are isoelectric points of the nano-particle, the substrate carrying only probe oligonucleotides, and the substrate carrying probe-target duplexes respectively. For purpose of illustration and not for purpose of limitation, the FIG. 7B shows binding energy of the colloidal particle to the substrate area carrying probe molecules only, E.sub.II, and probe and target molecules, E.sub.III, where each respective substrate area has the titration curve A-II and A-III respectively shown in FIG. 7A. The binding energy E.sub.II and E.sub.III are positive in solutions having pH in the range of pH>pI.sub.I and pH<pI.sub.III which corresponds to repulsion of particles from the surface. In solutions with pH in the range of pI.sub.III<pH<pI.sub.II the binding energy E.sub.III is negative and the binding energy E.sub.II is positive, which corresponds to binding particles to probe-target duplexes (e.g., E<0) and repulsion from the substrate surface carrying probe molecules only (e.g., E>0).

By reviewing energy diagram similar to that shown in FIG. 7B one skilled in the art can identify optimal reaction conditions at which nano-particles bind only to the substrate when target molecules are present on the substrate surface and said particles are repelled from the substrate when target molecules are not present. The factors which have significant importance for achieving the optimal performance of the detection method of present invention include the composition and density of ionizable chemical groups of the probe molecules and the substrate surface, the composition and density of ionizable chemical groups tethered on nano-particle surface, the size of nano-particle, the reaction pH, the composition and ionic strength of the reaction solution, the temperature and reaction time. In the preferred embodiment of the method the selection of the solution pH according to pI.sub.III<pH<pI.sub.II allows to achieve detection of the target molecules having minimum or no signal contribution from the probe molecules on the substrate. The range pI.sub.III<pH<pI.sub.II is determined by composition and density of ionizable groups of the substrate core material, the layers of material(s) on the substrate surface as well as by composition and density of the ionizable groups of the probe molecules.

Those of ordinary skill in the art will recognize that for predetermined group of probe and target molecules the proper selection of the substrate often allows to achieve a desirable pH range of pI.sub.III<pH<pI.sub.II. In addition, the composition of probe molecules and the use of chemical modification of probe molecules allow achieving a broader pH range of pI.sub.III<pH<pI.sub.II. Specific examples include, but not limited to, the use of aptamers for detection of proteins, the use of Peptide Nucleic Acids (PNA) for DNA and RNA detection, and the use of chemically modified oligonucleotides for DNA and RNA detection (e.g., aminopurine-, 5-methyl-, 5-nitroindole, deoxyinosine, deoxygenin, deoxyuridine, Uni-Link amino- and others oligonucleotide modifiers known in the art). In this example aptamer probe molecules are the preferred probe agents for detection of targets composed of amino-acids and chemical substances other than nucleic acids (e.g., for detection of target proteins, antibodies, glycoproteins, carbohydrates, hormones. etc.). The Peptide Nucleic Acid molecules are the preferred probe agents for detection targets composed of nucleic acids (e.g., DNA and RNA). Here, those skilled in the art will recognize that the difference of chemical composition of the probe and target molecules can be used to achieve the broader pH range in which nano-particles selectively bind to the substrate areas carrying the target molecules and do not bind to the substrate areas carrying only the probe molecules.

Now considering oligonucleotide probe for detection nucleic acids, an additional examples of probe modifications for broader pH range for selective target detection include, but not limited to, nucleotide analogs which contains some type of modification to either the base, sugar, or phosphate moieties. Modifications to the base moiety would include natural and synthetic modifications of nucleic bases such as uracil-5-yl, hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Sugar modifications include following modifications at the 2′ position: OH; F; O-, S-, or N-alkyl; O-, S-, or N-alkenyl; O-, S or N-alkynyl; or O-alkyl-O-alkyl, wherein the alkyl, alkenyl and alkynyl may be substituted or unsubstituted C1 to C10, alkyl or C2 to C10 alkenyl and alkynyl. 2′ sugar modifications also include but are not limited to —O[(CH.sub.2)n O]m CH.sub.3,—O(CH.sub.2)n OCH.sub.3, —O(CH.sub.2)n NH.sub.2, —O(CH.sub.2)n CH.sub.3, —O(CH.sub.2)n —ONH.sub.2, and —O(CH.sub.2)nON[(CH.sub.2)n CH.sub.3)].sub.2, where n and m are from I to about 10.

Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can contain inverted polarity such as 3′-5′ to 5′-3′ or 2′-5′ to 5′-2′. Many other base modifications can be found for example in U.S. Pat. Nos. 3,687,808; 4,845,205; 5,432,272; and 5,681,941, which detail and describe a range of base modifications. Each of these patents is herein incorporated by reference in its entirety, and specifically for their description of base modifications, their synthesis, their use, and their incorporation into oligonucleotides and nucleic acids.

### EXAMPLE II

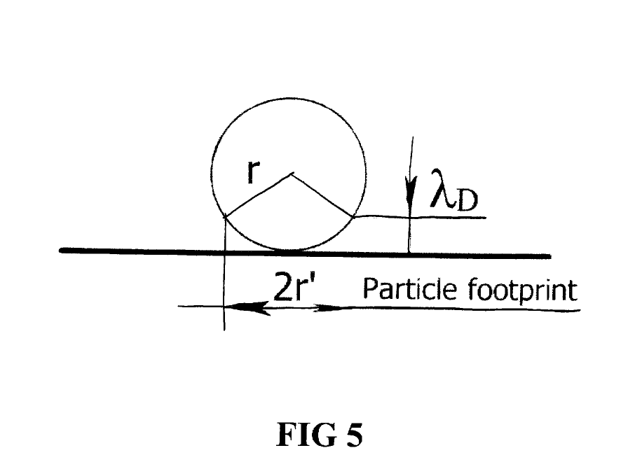

In one embodiment of the method of present invention the specific recognition of target molecules is achieved through the difference of the sign and local density of electric charge on the substrate surface due to presence of probe and target molecules. Binding energy of the nano-particle and the substrate is determined by a net electric charge inside the area on the substrate covered by nano-particle's “footprint” as illustrated in FIG. 5 and FIG. 6. In plurality of preferred embodiments the size of particle footprint is selected to be about the size of target molecule, and preferably, much larger than the size of corresponding probe molecule tethered on the substrate surface. For DNA targets and synthetic nucleotide probes the size of probe and target molecules bound to the substrate often is in the range of from about 2 nm to 1000 nm. Accordingly, the optimal size of the footprint of particle for DNA detection is preferably larger than 2 nm and most preferably is smaller than 10 microns. It is appreciated that the size of the particle's footprint is generally determined by a combination of two factors: (1) the physical size of the particle and (2) the distance on which an electric charge in solution is screened by solution's free ions (e.g., Debye length). For a particle of predetermined size the shorter the screening length in solution, the smaller the size of the particle's footprint. The screening length is decreasing with increasing the concentration of free ions in solution. For nano-particles larger than 2 nm the optimal size of particle's footprint can be set by adjusting the solution ionic strength. Considering 250-nm particles as an example, the desirable particle footprint for detection target DNA and proteins can be achieved in solutions having the concentration of free ions in the range of from 0.001 mM to 100 mM and most preferably in the range of from 0.01 mM to 10 mM.

Yet, another important parameter, which defines the attraction or repulsion of nano-particle from the substrate, is the solution pH. As has been discussed hereinabove, at proper selection of pH it is possible to achieve different sign of electric charge inside the particle footprint area on the substrate depending on the presence or absence of target molecules on the substrate surface.

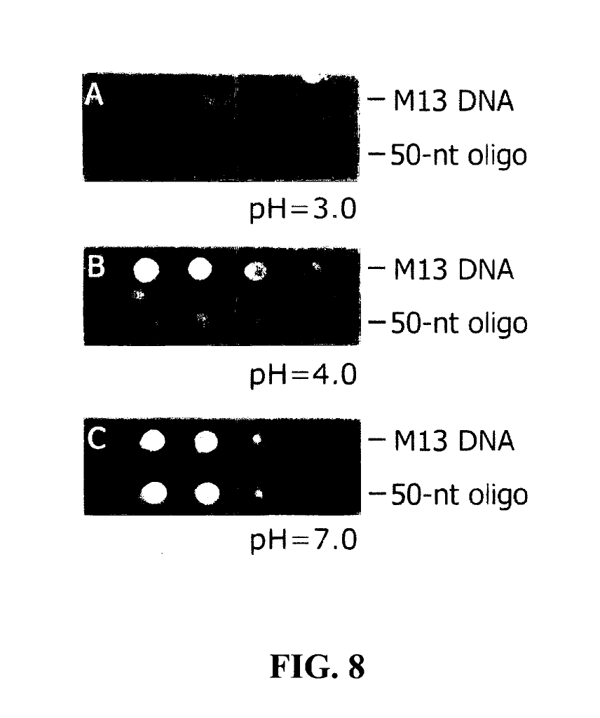

Now, presenting an example of experimental procedure for selecting solution pH, the method is demonstrated for selective detection of target 7,200-base long M13 phage ssDNA molecules. The target M13 ssDNA and 50-base long synthetic oligonucleotide probe have been immobilized on surface of Corning Ultra-GASP slide (Cat. No. 40016, Corning Life Sciences, MA). The probe and the target molecules have been immobilized on the substrate surface at the density of 10, 3.3, 1.1, and 0.34 ng/mm.sup.2 in array of total eight spots, where each spot carrying only probe or only target molecules. The nucleotides have been immobilized using Coming's Pronto! microarray printing reagents following the manufacturer's protocol (Pronto! Cat. No. 40028, Corning Life Sciences, MA). A solution of 250-nm cationic gold particles (AuroGene, Cat. No. AG-14. Sci-Tec, Inc., TN) at concentration of 1.4×10.sup.8 particles/ml has been prepared in solutions with different pH values and ionic strength adjusted by addition of HCl and NaCl. The concentration of Cl.sup.minus in solution was kept constant at 2 mM. The substrate with probe and target nucleotide has been developed for 15 min in 5 ml solution of the cationic gold particles at solution pH of 3.0, 4.0, and 7.0. The substrate subsequently was washed in distilled deionized water, dried by centrifugation and scanned by a flatbed scanner operating in dark-field detection mode. The image of the substrate surface with nano-particles bound to the surface is shown in FIG. 8. Consistently with the FIG. 7B and disclosure hereinabove, no binding of nano-particles observed in FIG. 8A at low pH, e.g., at pH=3.0. At pH=4.0 nano-particles bind selectively to the spots carrying target 7,200-nt DNA molecules with virtually no binding observed in spots carrying 50-nt probe oligonucleotides. At pH=7.0 the nano-particles bind with about the same efficiency to the probe and target molecules tethered on the substrate. In this example, the solution at pH=4.0 provides conditions for selective detection of target molecules on the substrate without undesirable detection of probe molecules present on the same substrate. Importantly, the target-selective detection in this embodiment is achieved without any modification/labeling of target molecules and without using sequence-specific agents or antibodies attached to nano-particles as would be required by methods known in the art for discriminating targets by sequence or by employing antibody-antigen interaction for recognition of target molecules.

### EXAMPLE III

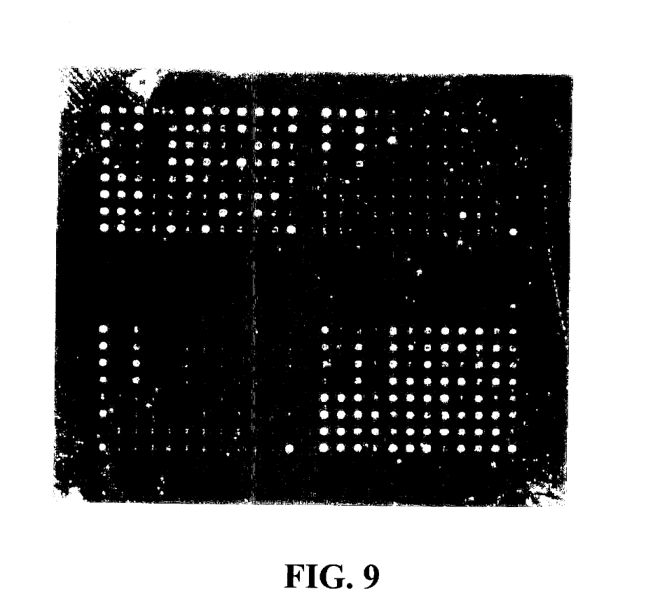

Yet in another example of the method illustrated in FIG. 9, a solution of 8-nm iron oxide particles has been used to detect latent pattern of hybridized nucleic acids on microarray surface. A microarray of 96 human synthetic oligonucleotide probes (human 96-gene sampler set. Illumina, Inc.) have been immobilized on surface of Corning Ultra-GASP slide (Cat. No. 40016, Corning Life Sciences, MA). The probe molecules have been immobilized using Corning's Pronto! microarray printing reagents following the manufacturer's protocol (Pronto! Cat. No. 40028, Corning Life Sciences, MA).

The microarray consisting of four blocks, two blocks printed at probes concentration of 2 mM and two block printed at probes concentration 20 mM, and where each block carrying 96 array spots, the microarray has been hybridized with a mixture of oligonucleotides from SpotCheck microarray slide quality control kit (Genetix, MA, USA) following Genetix hybridization protocol. After stringency wash, the microarray was dried by centrifugation.

The developing solution of 8-nm iron oxide particles (Alfa-Aesar, CAS#1309-37-1) at concentration of 0.5 mg/ml has been prepared in 5 mM Tris-HCl buffer at pH =8.0. The substrate carrying the latent pattern of hybridized oligonucleotides on the substrate surface have been developed by dipping the substrate into 5.0 ml of the iron oxide solution for 15 min. The substrate was subsequently washed in distilled deionized water, dried by centrifugation and scanned by Epson 3200 flatbed scanner operating in dark-field detection mode. The image of the detected nucleotide spots is shown in FIG. 9. The intensity of spots in FIG. 9 is the function of the amount of probe and target nucleotides bound to the surface at the corresponding array spot.

Importantly, in this example of the method of present invention the use of metal oxide nano-particles (e.g., iron oxide) has been demonstrated for detection of molecular structures on solid support without any modification target molecules and without coating nano-particles with nucleotide recognition agent(s) such as nucleotides, proteins, antibodies, or any other biopolymers. In this example the method of present invention eliminates the element of previous art, e.g., the use of nucleotide, protein or antibody coating of nano-particles for sequence-specific recognition of target molecules.

### EXAMPLE IV

In order to provide better understanding of the embodiment for using a powder of nano-particles for visualization and quantification biopolymers here an example of using 8-nm iron oxide particles will be presented. It is appreciated that in the method of present invention powder of nano-particles of other materials can be used including oxides carbides, nitrides, borides, chalcogenides, metals, alloys, and mixtures thereof.

For practicing method of present invention a latent pattern of molecular structures is produced on a substrate surface. One example of the substrate with the latent pattern of molecular structures is a microarray of DNA or synthetic oligonucleotides hybridized with target molecules, which target molecules bound to the homologous probes on microarray surface.

Another example of the substrate with the latent pattern of molecular structures is a microarray of probe proteins/antibodies to which target proteins, antibodies, glycoproteins. DNA, RNA, aptamers and similar molecular structures can be bound by exposing the microarray to a sample substance.

Yet, another example of said latent pattern of molecular structures is microarrays of probe molecules bind to targets including one or all of the following: proteins, antibodies, glycoproteins, metabolic products, DNA, RNA, aptamers and similar molecular structures.

One common feature of all methods of producing latent pattern of molecular structures is that the pattern of molecular structures on the substrate is not easily detectable optically or using other techniques since no labels, such as fluorescent, radioactive, or other labels or labeling chemical group, were incorporated neither into the probe, neither into the target molecules.

To illustrate an embodiment in which a powder of nano-particles is used for detection nucleic acid molecules, a latent pattern of 60-nt synthetic oligonucleotides was produced on UltraGASP microarray slide (Corning Life Sciences). Two oligonucleotides having different sequences shown in Table I have been spotted using solution of oligonucleotides Oligo 1 and Oligo 2 at concentration of 5.0, 2.5. 1.25, 0.61, 0.35, 0.18. 0.09, and 0.045 mu.g/ml.

TABLE I

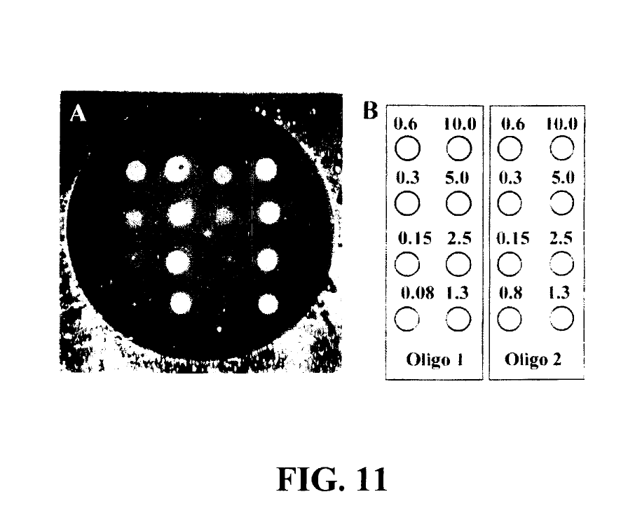

Synthetic oligonucleotides spotted on
microarray shown in FIG. 11.

| | |
|---|---|
| No. | 5′-3′ probe sequence |

| | |
|---|---|
| Oligo 1 | 5′-CGAAAGGGCCTCGTGATACGTAGGTTAATGTCATGATAA |
| | TAATGGTTTCT-3′ |
| Oligo 2 | 5′-AGGGTTTTCCCAGTCACGACGTTGTAAAACGACGGCCAG |
| | TGCCAAGCTTG-3′ |

After deposition of oligonucleotides, the microarray slide was kept overnight in a chamber at 75% humidity, and subsequently washed and UV-crosslinked at 600 mJ by UV Stratalinker 1800 (Stratagene, TX).

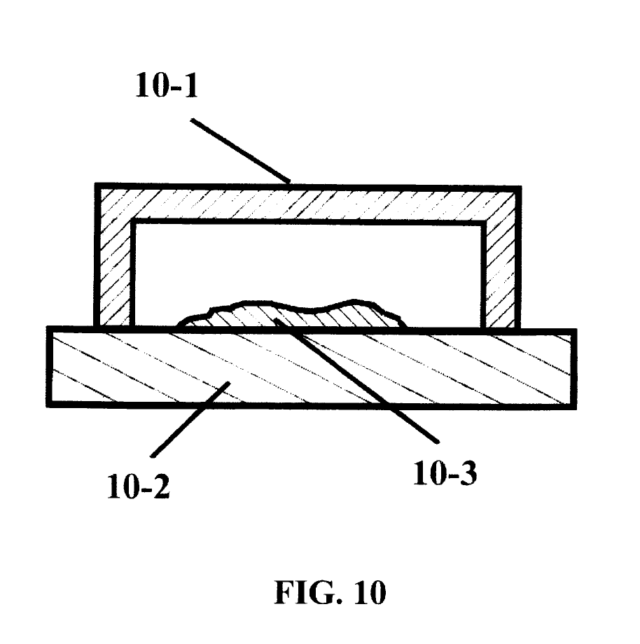

The pattern of the printed oligonucleotide spots was visualized using gamma iron oxide particles (Alfa-Aesar, CAS#1309-37-1). The substrate was exposed to the powder of nano-particles using chamber set up illustrated in FIG. 10. Now referring to the FIG. 10, the chamber from a commercial source (CoverWell.sup.™, Cat. No. GBL050418: 20 mm diameter×0.5 mm depth, Schleicher & Schuell) was partially filled with a powder of 8-nm iron oxide particles (Alfa-Aesar. CAS# 1309-37-1) The chamber was attached to the substrate through adhesive layer of the chamber's gasket, thereby forming peel-and-stick leak-proof enclosure on the substrate surface.

The chamber was filled with approximately 7 mg of the iron oxide powder. The microarray slide with the chamber attached was placed on the top of magnetic stirrer (IKA Works, Inc. Wilmington, N.C.). The steering speed of the magnetic stirrer was set to about 150 rpm. The magnetic field of the stirrer causes particles to group together and, following the magnetic field of the stirrer, form a rotating “swarm” of nano-particles moving on the substrate surface. To move particles all over the latent pattern on the substrate surface the substrate with the chamber attached was moved manually in an irregular pattern in the plane of the substrate surface. This movement force the “swarm” of the particles to move over the substrate surface thus allowing each and every spot of the substrate carrying latent pattern of oligonucleotides to interact with the particles for at least a short period of time. The treatment continued from 15 to 60 sec. The chamber was removed (e.g., pilled away) and the substrate was cleaned for 5-15 sec in a dust-free flow of compressed air.

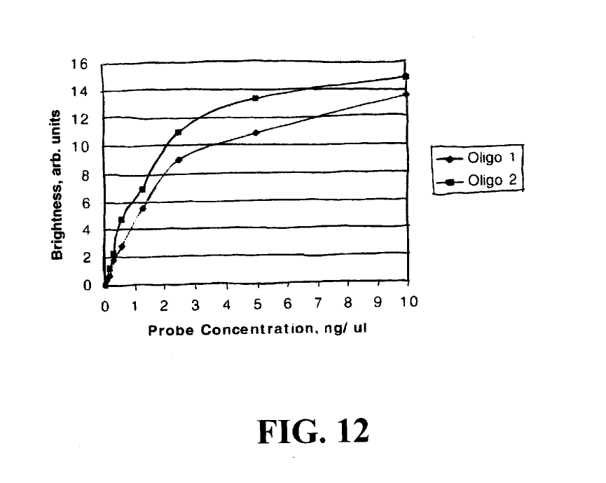

The array slide was placed in Epson Photo Impression 3200 flatbed scanner and image was captured in dark-field detection mode. The image is shown in FIG. 11A. FIG. 11B shows array layout and concentration of spotting solution in corresponding array spots. Average brightness of individual spots in FIG. 11A was measured using AuroGene 2.20 image acquisition and analysis software (Sci-Tec, Inc Knoxville, Tenn.). The brightness values are plotted in FIG. 12 vs. the amount of oligo deposited on the array surface. The data in FIG. 12 illustrate the method of present invention for quantitative measurement the amount of molecular structures present on solid substrate.

Importantly, in this example of the method of present invention the use of metal oxide nano-particles (e.g., iron oxide) has been demonstrated for detection of molecular structures on solid support without any modification of target molecules and coating nano-particles with nucleotide recognition agent(s) such as nucleotides, proteins, antibodies, or any other biopolymers for specific recognition of target molecules. Furthermore, binding of nano-particles to the substrate surface carrying latent pattern of molecular structures was carried out using powder without using any liquid phase reagents.

In this example the method of present invention eliminates the element of previous art, e.g., the use of nucleotide, protein or antibody coating of nano-particles for recognition and detection of target molecules and the use of solutions to carry out interaction of detection reagents and target molecules.

### EXAMPLE V

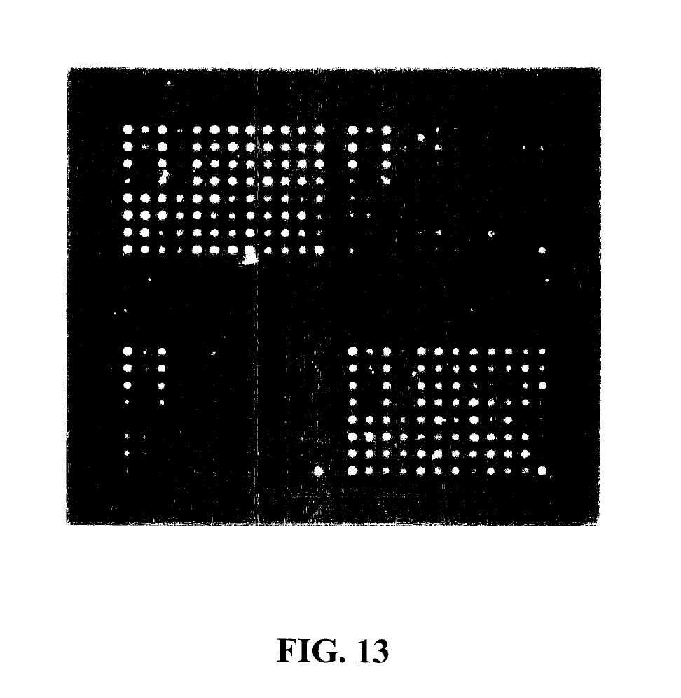

Yet in another example of the method illustrated in FIG. 13, a powder of 8-nm iron oxide particles has been used to detect latent pattern of hybridized nucleic acids on microarray surface. A microarray of 96 human synthetic oligonucleotide probes (human 96-gene sampler set, Illumina, Inc.) have been immobilized on surface of Corning Ultra-GASP slide (Cat. No.40016, Corning Life Sciences, MA). The probe molecules have been immobilized using Corning's Pronto! microarray printing reagents following the manufacturer's protocol (Pronto! Cat. No. 40028, Corning Life Sciences, MA).

The microarray consisted of four blocks, two blocks printed using oligonucleotides at concentration 20 mM and two blocks printed using oligonucleotides at concentration of 20 mM., each block carrying 96 array spots. The microarray has been hybridized with a mixture of oligonucleotides from SpotCheck microarray slide quality control kit (Genetix, MA, USA) following Genetix hybridization protocol. After stringency wash, the microarray was dried by centrifugation.

The pattern of the printed oligonucleotide spots was visualized using 8-nm iron oxide particles (Alfa-Aesar, CAS#1309-37-1). The microarray with hybridized target oligonucleotides was exposed to the powder of nano-particles using chamber set Up illustrated in FIG. 10. Now referring to the FIG. 10, the chamber from a commercial source (CoverWell.sup.™, Cat. No. GBL050418: 20 mm diameter×0.5 mm depth, Schleicher & Schuell) was partially filled with a powder of 8-nm iron oxide particles (Alfa-Aesar, CAS#1309-37-1) The chamber was attached to the substrate through adhesive layer of the chamber's gasket, thereby forming peel-and-stick leak-proof enclosure on the substrate surface.

The chamber was filled with approximately 7 mg of the iron oxide powder. The microarray slide with the chamber attached was placed on the top of magnetic stirrer (IKA Works, Inc. Wilmington, N.C.). The steering speed of the magnetic stirrer was set to about 150 rpm. The magnetic field of the stirrer causes particles to group together and, following the magnetic field of the stirrer, form a rotating “swarm” of nano-particles moving on the substrate surface. To move particles all over the latent pattern on the substrate surface the substrate with the chamber attached was moved manually in an irregular pattern in the plane of the substrate surface. The treatment continued for about 15 sec. The chamber was removed (pilled away) and the substrate was cleaned for 5 sec by a flow of compressed air.

The array slide was placed in Epson Photo Impression 3200 flatbed scanner and image was captured in dark-field detection mode. The image is shown in FIG. 13 and is consistent with the image in FIG. 9 obtained by developing microarray in solution of iron oxide particle. The image in FIG. 13 therefore illustrates the method of present invention for visualization and quantification molecular structures on solid support.

Importantly, in this example of the method the use of metal oxide nano-particles (e.g., iron oxide) has been demonstrated for detection of molecular structures on solid support without any modification or coating nano-particles with nucleotide recognition agent(s) such as nucleotides, proteins antibodies, or any other biopolymers. Furthermore, binding of nano-particles to the substrate surface is demonstrated by using powder of nano-particles without using any liquid phase reagents to maintain interaction of nano-particles and target molecules on the substrate surface.

### EXAMPLE VI

### Detection of Poly-L-Lysine on a Polymer Substrate

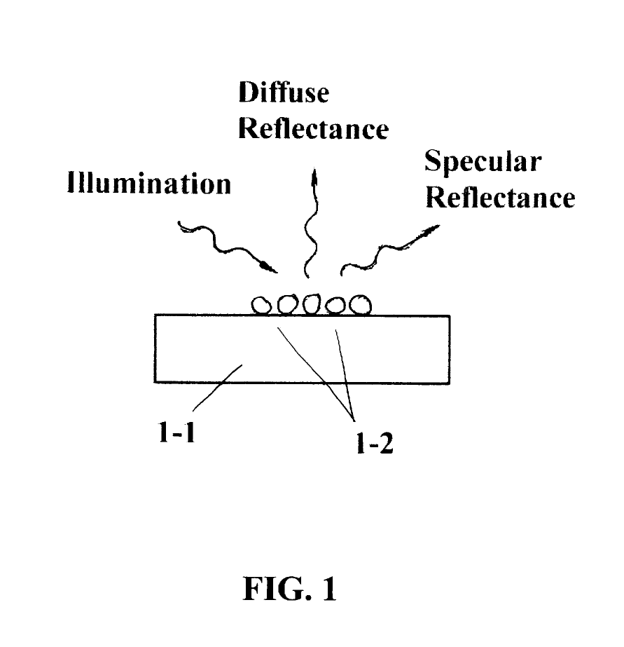

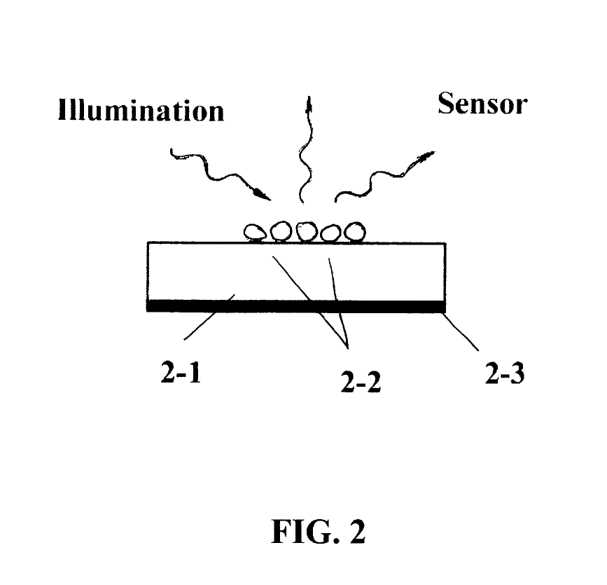

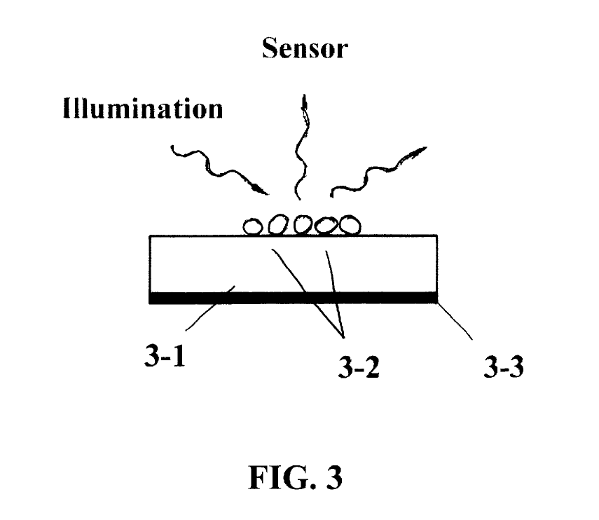

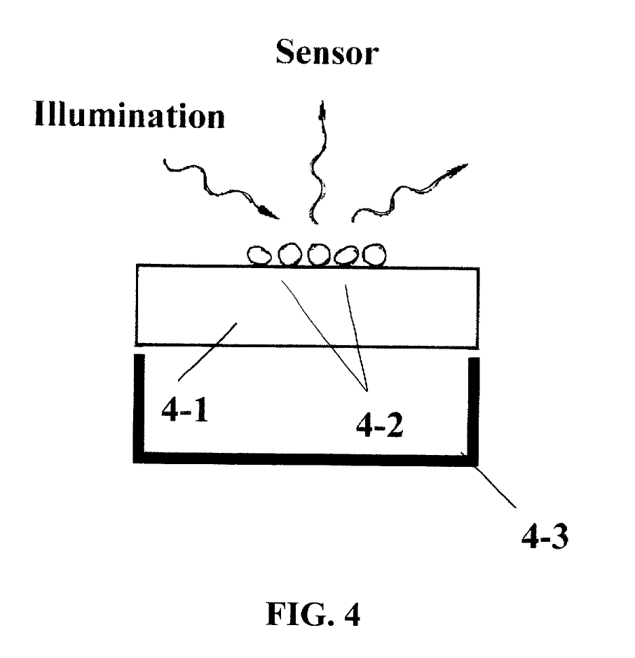

When nano-particles on a surface are illuminated by external light source, the light is partially absorbed, specular reflected and diffusely reflected by nano-particles bound on the substrate surface, as illustrated in FIG. 1A. In the visible spectral range the net reflected portion of the light normally dominates over the portion of the light absorbed by nano-particles when the size of particles is 50 nm or less. Therefore, more sensitive detection of gold particles usually can be achieved by detecting reflected and scattered light vs. the measurement of the absorption. The exact ratio of the diffusely reflected component to the specular light component varies vs. the size of the particles. The intensity of diffuse reflected light often dominates over the reflected light when the size of metal particles is in the range of larger than 20 nm.

An optical flatbed scanner is particularly preferable for capturing light diffusely reflected by a surface. Two important components of the scanner are light source for illuminating the surface and a linear CCD element, which capture scattered light at some angle to the direction of illumination. In most commercially available scanners special measures are taken to minimize the specular reflected component captured by CCD element. To acquire image, the light source and CCD element move along the surface and capture pattern of the diffuse reflectance on the surface. This operation mode, e.g. front illumination mode normally is used to capture images of paper documents. Most scanners are also equipped with a white lead screen and some scanners are equipped with additional diffuse light source for operating in a backside illumination mode. which is optimal for capturing prints produced on transparent substrates. In the back-illuminating mode light passes through the substrate, such as slide or film. Light captured by scanner sensor represents the sum of the absorbance and specular reflectance of the substrate. The backside illumination mode (e.g., bright-field detection mode) is particularly useful for detecting light-absorbing regions on the surface of transparent substrates and with the method of present invention can be used for detection absorption of nano-particles. However, we found that 50 nm, 100 nm and 250 nm metal particles provided a stronger signal and better signal-to-noise ratio when detected in front-illuminating mode by detecting diffusely reflected light (e.g., dark-field detection mode).

To select a scanner for using with the method of present invention a number of parameters have to be taken into consideration, including: 1) Optical or true resolution, which for currently available commercial scanners is in the range from 1200 dpi to 6400 dpi; 2) The ability to export high dynamic range images, i.e, to export 16-bit gray or 48-bit color images; 3) The ability to operate either in front- or back-side illuminating mode; and 4) The rate of sending data to computer. In this example, the Epson Perfection 3200 (Seiko Epson Co.) flatbed scanner has an optical resolution up to 6400 dpi, can export 48-bit color images, can operates both in front- and back illuminating modes and provides data transfer rate up to 400 Mb/s. To take advantage of the capability of the scanner to make quantitative measurements, a TWAIN-compatible software was developed which allowed multiple scans and capture of any pre-defined number of scans of a specified region on the substrate. The multiple scans were used to accumulate signal and improve Signal-to-Noise (S/N) ratio for improving overall detection sensitivity. Different modes of capturing data were implemented for detection of nano-particles on opaque polymer substrate, blackened glass substrates, and transparent substrates in combination with light absorbing screen placed behind the substrate. In particular, the techniques include multiple scanning of the substrate surface and exploiting the benefit of capturing and processing high amount of information contained in high-resolution images.

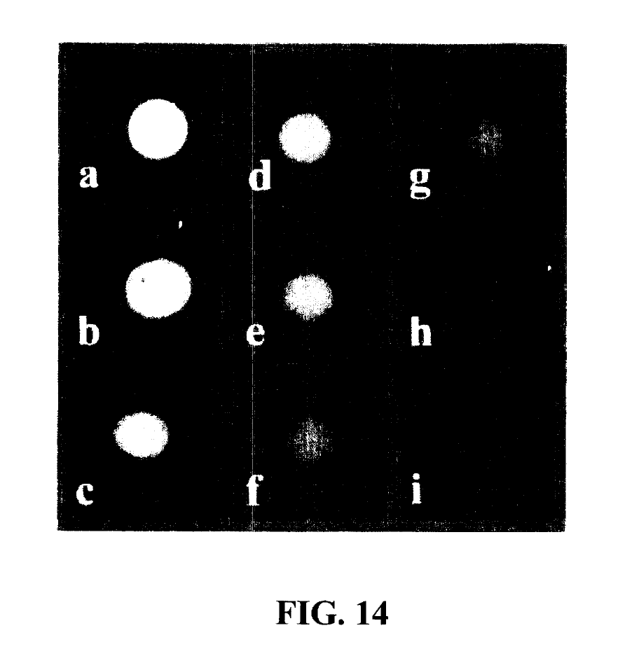

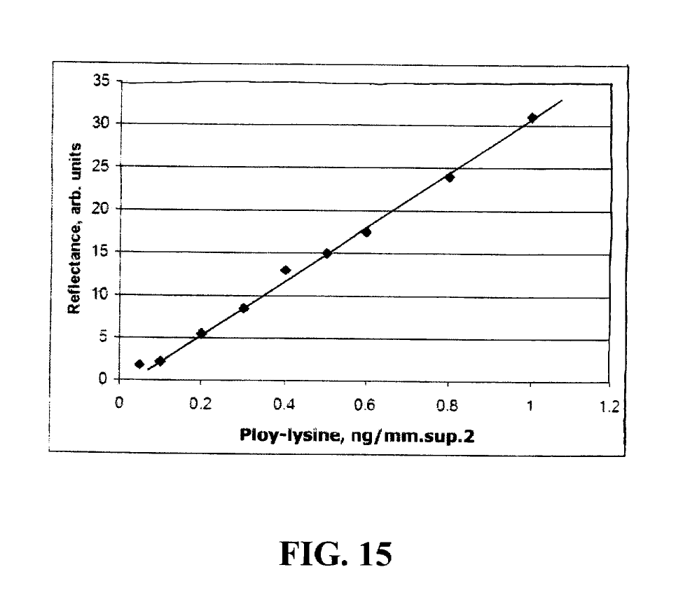

FIG. 14 show example of binding 250-nm gold particles (BBInternational, UK) on the surface of opaque Mylar film. In this illustration of the method of present invention a latent pattern of spots of poly-L-lysine was first produced on the substrate by pipetting 1 .mu.l of poly-L-lysine solution at concentrations (a) 1 ng/.mu.l, (b) 0.8 ng/.mu.l, (c) 0.6 ng/.mu.l, (d) 0.5 ng/.mu.l, (e) 0.4 ng/.mu.l, (f) 0.3 ng/.mu.l, (g) 0.2 ng/.mu.l, (h) 0.1 ng/.mu.l, and (i) 0.05 ng/.mu.l. The latent pattern was visualized by developing the substrate in solution of 250 nm gold particles at concentration 3.6.times. 10.sup.8 ml.sup.−1. The development was carried out at room temperature by dipping the substrate for 15 min into the solution of gold colloid. FIG. 15 shows plot of intensity of corresponding spots vs. the amount of poly-L-lysine on the substrate surface for array image shown in FIG. 14. The data in FIG. 15 illustrates the way of quantitative measurements of the amount of molecular structures on substrate surface by measuring the intensity of light scattered by particle bound to the surface.

### EXAMPLE VII

### Image Capturing Techniques for Increasing Signal-to-Noise (S/N) Ratio for Achieving Higher Detection Sensitivity

Enhancing S/N by averaging multiple scans: Averaging multiple scans increases S/N as the squareroot of the number of scans. This was observed and verified by capturing multiple scans of the image shown in FIG. 14. S/N was measured as the ratio of the average amplitude of the light diffusely reflected at the center of a spot labeled by gold to the average variation of the background signal in the close proximity to the spot. The same type of dependence of the S/N vs. number of scans was observed for 24-bit color images and 48-bit color images acquired using Epson Perfection 3200 scanner. For the same number of acquired scans S/N ration was higher for red and green component of the image and somewhat lower for the blue component of the color image of the gold particles. The last result is consistent with the yellowish color of the gold particle, since the red and green component of this color is higher than the corresponded blue component. For this reason, processing the red and green component of the image of gold particles and discarding the blue component of the image can usually provide a higher S/N ratio.

Enhancing S/N by reducing image size: Capturing an image at high-resolution and converting it to a lower resolution image often increases S/N. For instance, when the size, i.e. width and height, of the image is reduced twice, each block of 2.times.2, i.e., total of 4 pixels of the original image is “squeezed” into one pixel of the lower resolution image. When reducing the size of the image the amplitude of pixels of smaller image normally is calculated as average amplitude of a group of pixels from the original image. In the last example, reducing the size of the image twice can cause same effect on S/N as accumulating and averaging four lower resolution images. i.e., it increases S/N twice. Capturing image shown in FIG. 14 at resolutions of 800, 1200, 1600, 3200, and 6400 dpi and then reducing the size of the image to 600 dpi has confirmed this conclusion. In tests, the S/N was measured as the ratio of the amplitude of the light diffusely reflected at the center of a spot labeled by gold to the average variation of the background signal in the close proximity to the spot. As was discussed herein above, the S/N increases linear vs. the factor to which the size of the image was reduced and for image captured at 6400 dpi reducing the size to 600 dpi causes about 16-fold increase of S/N.

### EXAMPLE VIII

### Opaque and Blackened Substrates for Detection of Molecular Structures of Interest

Different types of substrates were employed with the method of present invention for immobilization of biopolymer molecules, including a glass and fused silica slides, polycarbonate polymers and Mylar.™. polymer film. In tests, to minimize undesirable background signal from light passed through the substrate and reflected back by scanner lead or environment, a back surface of a transparent substrate was blackened by acrylamid-based paint (see FIG. 1B and FIG. 1C). Also were investigated substrates of polymer films known for production of magnetic floppy diskettes (Immation Co., MN). The surface of the polymer substrate can be activated for immobilization of probe molecular structures using known chemistries for modification polymers. In this example of embodiment of the method the polymer substrate was activated for immobilization of DNAs and protein molecules by treatment in solution of poly-L-lysine for.

For immobilizing biopolymers on lysine coated surface the substrate was treated for 15 minutes in 0.1% solution of poly-L-lysine (Sigma-Aldrich. Cat. No. P8920), followed by washing in a distilled water. The substrate was dried in a flow of compressed filtered air. Amino-modified substrates for immobilizing biopolymers were prepared by exposing polymer or glass substrates to 1% solution of .gamma.-aminopropyltriethoxylsilane in alcohol for 1 hour. Substrates were subsequently washed in distilled water, dried at room temperature and stored in desiccated container at room temperature.

### EXAMPLE VIII

### Immobilization and Visualization of DNA and Proteins

Protein A (Cat. No. P6031), Human Immunoglobulin G from Serum (Cat. No. 14506). Bovine Serum Albumin (Cat. No. A7511), and single stranded 7,229 bases long M13mp8 Phage DNA (Cat. No. D8410) were purchased from Sigma. MO. A set of 11 monoclonal antibodies specific for DNA repair pathways was purchased from BD Biosciences (Cat. No. 611432). A 70 bases long synthetic oligonucleotides of different sequences with varied A-T vs. C-G composition were synthesized and PAGE-purified by AlphaDNA (Montreal, Quebec, Canada). For non-covalent immobilization (passive absorption) on substrate surface stock solution of each biopolymer was prepared at concentration of 100 ng/.mu.l in distilled deionized water or, alternatively, in 0.3 M sodium acetate buffer (Cat. No. S-7899, Sigma, MO). For spotting corresponded biopolymers on substrate surface, freshly prepared stock solutions were diluted to a lower concentration of 10 ng/.mu.l and 1 ng/.mu.l as needed.

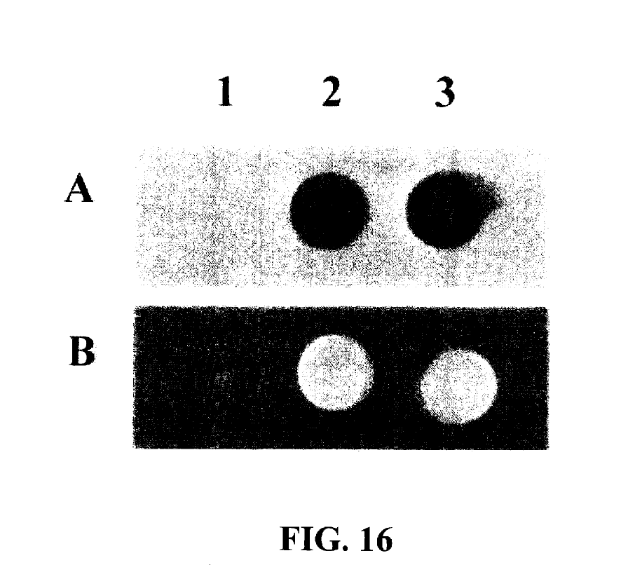

Detecting DNA: A single-stranded 7,200-base long Phage DNA (M13mp8) was immobilized on lysine coated Mylar substrate by pipetting 1 .mu.L of each of three dilutions containing 100, 10, and 1 ng/.mu.L of the DNA. To maintain absorption from solution and preventing spots from drying the substrate was incubated at room temperature overnight in humidified chamber. The substrate was thoroughly washed in distilled water to remove spotting solutions and unbound DNA molecules. The substrate with latent pattern of DNA spots was exposed for 30 minutes to solution of negatively charged 250 nm gold particles at concentration 3.6.times.10.sup.8 particles/mi. FIG. 16A shows image of the developed substrate captured by scanner. The substrate is uniformly covered by gold particles except for the spots where DNA was spotted at concentration 10 and 100 ng/.mu.l. The absence of gold particles in these spots is consistent with the fact that DNA is carrying a net negative electric charge, which can repel negatively charged gold particles. To confirm that unstained spots in FIG. 11A indeed are caused by electrostatic repulsion of gold particles by negatively charged DNA, a solution of positively charged gold particles has been prepared by immobilizing poly-L-lysine on gold particles. Positively charged nano-particles were prepared by adding 80 p.mu.of 0.01% poly-L-lysine (Sigma-Aldrich) to 1 ml of gold colloid at concentration 3.6.times. 10.sup.8 particles/ml. The mixture was incubated at room temperature at constant shaking for 2 hours. To remove unbound poly-L-lysine the solution was centrifuged to precipitate nano-particles and the natant carrying the residual unbound poly-L-lysine was discarded. A pellet of nano-particles was resuspended in distilled deionized water to the original concentration of 3.6.times.10.sup.8 particles/ml and this colloidal solution was used to develop the latent pattern of the DNA molecules on the substrate. The development was carried out by dipping the substrate into the colloidal solution for 15 min at room temperature. When development was completed, the substrate was washed gently using distilled deionized water, dried by centrifugation and scanned using Epson Perfection 3200 flatbed scanner.

The positive charge on gold particles was confirmed first by applying to a lysine or amine coated substrate, on which no precipitation of gold particles on the substrate was observed. Next, the solution of positive gold particles was applied to the amino-modified polymer substrate carrying latent pattern of immobilized DNA as described herein above. The gold particles bound to sites where DNA was spotted. A corresponded image of the substrate is shown in FIG. 16B where the image resembles a “negative image” of the spots in FIG. 16A. This is consistent with the mechanism of ionic interaction of charged gold particles and molecular structures on the substrate surface.

### Reducing Gold Binding Capacity of Activated Surfaces and Competitive Labeling of Biopolymers on a Surface

In some cases it might be desirable to block or cap activated surface and prevent binding gold particles which otherwise occur all over the substrate surface. Different blocking reagents and chemistries were reported in the literature, including the use of acetic anhydride for blocking amine groups on an amine terminal spacer and glycine for blocking poly-L-lysine coated surfaces. In addition to the methods known in the art, in the method of present invention exposing activated surfaces to a solution of serum albumin (BSA) modifies and reduces binding capacity of the surface. By choosing an appropriate exposure time and by adjusting the concentration of the albumin molecules in solution it is possible to achieve a partial or complete blocking of the surface against precipitation of gold particles. The approach of blocking the surface by albumin has been used by the method of present invention for competitive labeling of molecular structures on a substrate surface. To illustrate this approach spots of poly-L-lysine were printed on amino-modified Mylar surface as presented herein above in Example I. When the substrate was developed in a colloidal solution of 250 nm negatively charged gold particles at a concentration of 3.6.tines.10.sup.8 particles/ml for 30 minutes, the substrate surface was saturated and uniformly covered by gold particles. Indeed, both polylysine molecules and amino groups are bound to gold particles and no polylysine spots on the surface could be identified. However when an amino-modified substrate with spots of polylysine was developed in a colloidal solution containing 3% of Bovine Serum Albumin, the average density of bound gold particles was significantly reduced everywhere except for the spots on the surface covered by poly-L-lysine. The spots can be easily identified and quantitatively characterized using the image captured by a scanner. Indeed, when both nano-particles and albumin molecules are added to the developing solution, the molecules and particles are competing for the bind sites on the substrate surface. Absorption rate of albumin on amino-modified surface may be higher then in the region covered by poly-L-lysine. This causes the surface to be blocked faster by albumin everywhere on the surface except for the spots covered by poly-L-lysine. The difference in the reaction rate is projected into the variation of the density of bound gold particles on the surface and can be used to discriminate spots of different biopolymers based on the difference in the rate of attaching nano-particles and an alternative binding agent such as BSA.

### EXAMPLE IX

Yet in another example of the method illustrated in FIG. 13, a solution of 250-nm cationic gold particles (Cat. No. AG14, Sci-Tec. Inc., Knoxville, Tenn.) has been used to detect latent pattern of hybridized nucleic acids on microarray surface. A microarray of 96 human synthetic oligonucleotide probes (human 96-gene sampler set, Illumnina, Inc.) have been immobilized on surface of Coming Ultra-GASP slide (Cat. No. 40016, Corning Life Sciences, MA). The probe molecules have been immobilized using Corning's Pronto! microarray printing reagents following the manufacturer's protocol (Pronto! Cat. No. 40028. Corning Life Sciences, MA).

The microarray consisted of four blocks, two blocks printed using oligonucleotides at concentration 2 mM and two blocks printed using oligonucleotides at concentration of 20 mM, each block carrying 96 array spots. The microarray has been hybridized with a mixture of oligonucleotides from SpotCheck microarray slide quality control kit (Genetix, MA. USA) following Genetix hybridization protocol. After stringency wash, the microarray was dried by centrifugation.

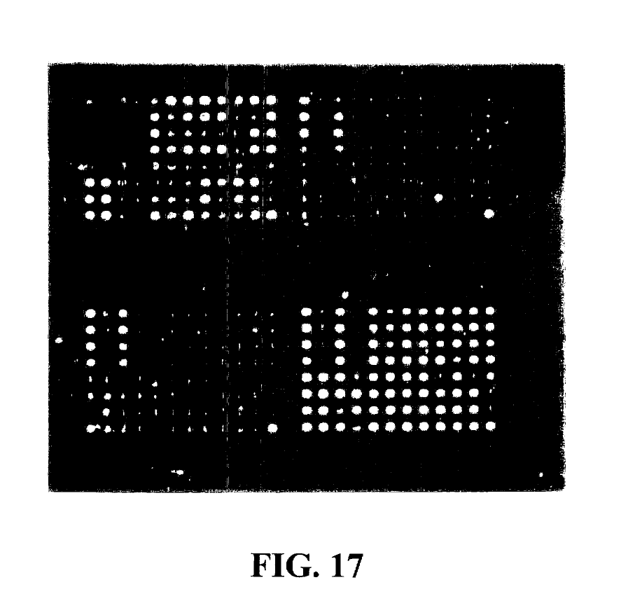

A solution of 250-nm cationic gold particles (AuroGene, Cat. No. AG-14, Sci-Tec, Inc., TN) at concentration of 1.4×10.sup.8 particles/ml has been prepared in 5 mM potassium biphthalate buffer at pH=4.0. The microarray has been developed by dipping the substrate for 15 min into 5 ml of the solution of cationic gold particles. The substrate subsequently was washed in distilled deionized water, dried by centrifugation and scanned by Epson 3200 flatbed scanner operating in dark-field detection mode. The image of the substrate surface with gold nano-particles bound to the surface is shown in FIG. 17 and is consistent with the images of the same type of microarray developed in solution of 8-nm iron oxide particles shown in FIG. 9 and with microarray developed using powder of 8-nm iron oxide particles shown in FIG. 13.

### EXAMPLE X

### Detecting Protein A and Immunoglobulin G

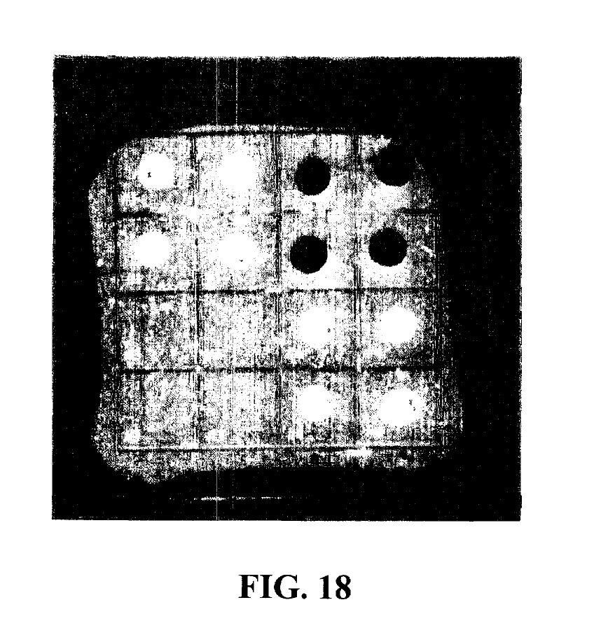

Different biopolymers may have different affinities to nano-particles. When immobilized on a surface, such biopolymers increase or reduce particle binding capacity of the surface and can be detected by measuring the amount of nano-particles bound on the surface. Two examples, which can illustrate this approach are detection of protein A and Immunoglobulin G (ImG). When immobilized through passive adsorption on amino-modified Mylar substrate, the first, protein A, decreases and the second, ImG, increase gold binding capacity of the substrate. The latent pattern of proteins on the substrate was prepared by spotting 1 .mu.l of 100 ng/.mu.l solution of Protein A and Immunoglobulin G in 0.3M sodium acetate buffer and by incubating the substrate overnight in humidified chamber. Next, part of the substrate carrying two spots of the Protein A (see spot #4 in FIG. 18) was incubated for 1 hour at room temperature in a solution of ImG at a concentration 100 ng/.mu.l. The substrate was washed in distilled deionized water, dried and developed in solution of 250 nm negatively charged gold particles at concentration 3.6.times.10.sup.8 particles/ml for 5 minutes. The relatively short developing time was used to avoid saturation and reduce the density of bound gold particles below the maximum gold binding capacity of the substrate. Under such conditions, the density of gold particles in the spot covered by ImG is higher than the background density (see spots #1 in FIG. 18) and the density in the spot covered by Protein A is lower than the background density of gold particles (see spots #2 in FIG. 18). The Protein A and ImG are capable to bind and form probe-target complex upon interaction. This can be observed in spots #4 in FIG. 18, where reducing of the gold binding capacity of the substrate due to immobilization of Protein A is overcome by increasing the binding capacity due to attachment of ImG at sites where Protein A is immobilized.

A set of dilutions was used to immobilize different quantity of ImG on substrate and determine the detection sensitivity for ImG. Consistently with what was previously observed for DNA and poly-L-lysine. ImG spots with density of 0.2 ng/mm.sup.2 were detectable and show S/N>3 in an image captured after a single scan of the substrate surface. An increase of binding capacity of the surface and similar detectable density of antibodies of about 0.2 ng/mm.sup.2 were observed for a set of 10 monoclonal antibodies specific for DNA repair pathways (BD Biosciences, CA, Cat. No. 611432).

It is appreciated that for detection of target proteins carrying various sign electric charge the substrate with the latent pattern of target proteins can be treated in solution of anionic detergent for period of time from 5 sec to 8 hours and the concentration of detergent solution in the range of from 0.01 mM to 1 M. One particular example of the treatment of the latent pattern of target proteins and antibodies is the treatment of bound target proteins in 1% SDS (Sodium dodecyl sulfate) solution. Ionic binding of the anionic detergent to the positively charged targets is known in the art for producing negatively charged detergent-target complexes, which said negatively charged complexes subsequently can be detected and quantified according to the method of present invention using cationic nano-particles. In this example the treatment of the latent pattern consisting of positively and negatively charged target molecules in a solution of anionic detergent creates negative charged pattern on the substrate, wherein all target molecules now can be visualized and quantified using positively-charged nano-particles.

### EXAMPLE IX

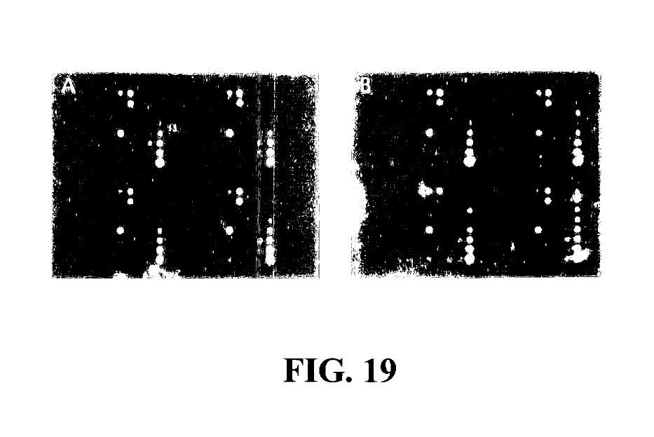

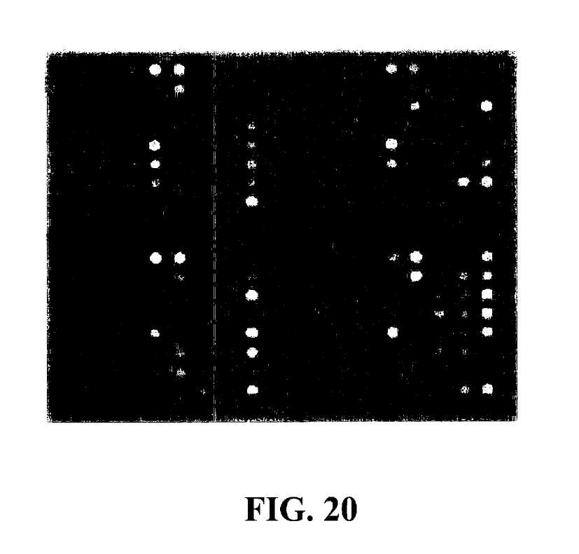

FIG. 19 and FIG. 20 show an example of using cationic nano-particles for detecting differential gene expression in paired RNA samples from human Jurkat cells stimulated by ionomycin/PMA.

T-cell activation is one of the most widely studied models of cellular response to exogenous stimulation. Ionomycin/PMA activation of signal transduction cascades resulted in gene expression pattern characteristic of the immune response. In this example of the method a small subset of 30 genes was identified which are commonly studied in T-cell activation: ACTB, CCL15, CCNA2, CD69, CEBPB, EGR1, EGR2, FOS, GAPD, GATA3, IFNG, ILIR1, IL2, IL2RA, IL6, IL8, JUN, JUNB, JUND, MHC2TA, NYC, NFKB1, PPIA, STAT1, YY1, FOG2, The abundance and the fold-change of these genes is expected to vary over one order of magnitudes. A set of 6 housekeeping genes and 3 negative control genes (pUC 18, alien and yeast) have been included into the set in Table 1. The synthetic oligonucleotide probes have been purchased from Operon Technologies (Alameda, Calif.). The list of genes and sequence of the corresponding probes on microarray are shown in Table 11.

TABLE II

Oligonucleotide probes for detection gene
expression of normal and ionomycin/PMA-
stimulated Jurkat cells.

| | | |
|---|---|---|
| No. | GENE | 5′-3′ probe sequence |

| | | |
|---|---|---|
| 1 | ACTB | 5′-ACATAATTTACACGAAAGCAATGCTATCACCTCCCCTG |
| | | TGTGGACTTGGGAGAGGACTGGGCCATTCTCC-3′ |
| 2 | CCL15 | 5′-GGAGGGGGCCTTGGCATCTTCTCTTTATGTCTCTGAGC |
| | | TGTGCCTTCGCCACCCCTTCTGGGTCACTCAG-3′ |
| 3 | CCNA2 | 5′-TCTCTTATTGACTGTTGTGCATGCTGTGGTGCTTTGAG |
| | | GTAGGTCTGGTGAAGGTCCATGAGACAAGGCT-3′ |
| 4 | CD69 | 5′-CCAGTAGTGCAAATGCATGAAGGGCTCTCACTGTTGGT |
| | | AGTCATTCAGCAATAAATAGTAAGTCCACGCC-3′ |
| 5 | CEBPB | 5′-TCGGGCAGCTGCTTGAACAAGTTCCGCAGGGTGCTGAG |
| | | CTCGCGCGACAGCTGCTCCACCTTCTTCTGCA-3′ |
| 6 | EGR1 | 5′-GTTTTCTTACATTCTGGAGAACCGAAGCTCAGCTCAGC |
| | | CCTCTTCCTTATTTTGCTCCCAAAGCCTCCCC-3′ |
| 7 | EGR2 | 5′-GGCAACCCATTTACATGCAGCCTTGTAACATTTGTCTA |
| | | CATCACACAAGGCGACCAAGGACACTTCCAAC-3′ |
| 8 | FOS | 5′-AGGCCTGGCTCAACATGCTACTAACTACCAGCTCTCTG |
| | | AAGTGTCACTGGGAACAATACACACTCCATGC-3′ |
| 9 | GAPD | 5′-GGTTGAGCACAGGGTACTTTATTGATGGTACATGACAA |
| | | GGTGCGGCTCCCTAGGCCCCTCCCCTCTTCA-3′ |
| 10 | GATA3 | 5′-GCATGTAGGCCTAGAAAAAGGCTCTCTGAAACCCTCAA |
| | | TGGCAACTGGTGAACGGTAACACTGATTGCCC-3′ |
| 11 | IFNG | 5′-ACACACAACCCATGGGATCTTGCTTAGGTTGGCTGCCT |
| | | AGTTGGCCCCTGAGATAAAGCCTTGTAATCAC-3′ |
| 12 | IL1R1 | 5′-GTCAAAGGAAGTTCACGGGGAACTAGGAATGTGTCTTC |
| | | TTCCTCCAGAATTCAACCCTTGGAAGATGGGG-3′ |
| 13 | IL2 | 5′-CAGCAGTAAATGCTCCAGTTGTAGCTGTGTTTTCTTTG |
| | | TTTTCTTTGTCGAACTTGAAGTAGGTGCACTG-3′ |
| 14 | IL2RA | 5′-CTTCTCAGGAAACGTACGCATTGATTTGCACCTTGTGT |
| | | GTCCACCTGTAAACATCAAATTAGTGCAGGCC-3′ |
| 15 | IL6 | 5′-CTGACCAGAAGAAGGAATGCCCATTAACAACAACAATC |
| | | TGAGGTGCCCATGCTACATTTGCCGAAGAGCC-3′ |
| 16 | IL8 | 5′-GCACTACCAACACAGCTGGCAATGACAAGACTGGGAGT |
| | | ATCAAACTAGGATTGTTAGTTCAATTAAAACT-3′ |
| 17 JUN | 5′-CACTGCAACCCCCCTTCCTCCAGCCTCCTGAAACATCG | |
| | | CACTAGCCTTTTGGTAAGCAATTCCATATAGAT-3′ |
| 18 | JUNB | 5′-GGGGCCAGCTCCGCCGCGATCGCCCCCTCTTCCCCTCC |
| | | CTGTTAAATACACAAATATATTATATTCAATA-3′ |
| 19 | JUND | 5′-GGCGTAACGAGACTTTACTGAAAACAGAAAACCGGGCG |
| | | AACCAAGGATTACAAACAGGAATGTGGACTCG-3′ |
| 20 | MHC2TA | 5′-ACCACCCTCTCTGGGCCCTTTCATTCTCTGCTATGGAC |
| | | TGAGTGGACCAGCTTGGATCAAAATCCTCAAA-3′ |
| 21 | MYC | 5′-GCTTTTGCTCCTCTGCTTGGACGGACAGGATGTATGCT |
| | | GTGGCTTTTTTAAGGATAACTACCTTGGGGGC-3′ |
| 22 | NFKB1 | 5′-AGTTAAATCGAGAATGATTCAGGCGGGCCGGCTCTCTG |
| | | AGCACCTTTGGATGCACTTCAGCTTCTGTCTT-3 |
| 23 | PPIA | 5′-GTCGAAGAACACGGTGGGGTTGACCATGGCTAATAGTA |
| | | CACGGTTTTCCTCGGCGGTGGCGTCTGCAAAA-3′ |
| 24 | STAT1 | 5′-CCAATACAGGCGCTCTGCTGTCTCCGCTTCCACTCCAC |
| | | TAGTTCATCATTAATCAGGGCATTCTGGGTAA-3′ |
| 25 | YY1 | 5′-TCTACAACTGAGCACCACTTTCTGTAACTGAACAGGCA |
| | | AAGAAATTACACTGAACATCAGCATCTGGCAG-3′ |
| 26 | FOG2 | 5′-CAGGTTCAGGATTAAGAAAATGGACGGAAACATACAGC |
| | | TACATACAAATGCAAAGCCTAGTGACTAAGAG-3′ |
| 27 | PUC18 | 5′-CGAAAGGGCCTCGTGATACGTAGGTTAATGTCATGATA |
| | | ATAATGGTTTCTTAGACGTCAGGTGGCACTTT-3′ |
| 28 | polyT | 5′-TTTTTTTTTTTTTTTTTTTTTTTTTTTTTTTTTTTTTT |
| | | TTTTTTTTTTTTTTTTTTTTTTTTTTTTTTTT |
| 29 | M13(+) | 5′-AGGGTTTTCCCAGTCACGACGTTGTAAAACGACGGCCA |
| | | GTGCCAAGCTTG-3′ |
| 30 | Neg | Printing buffer only |

A focused microarray of 120 spots carrying 30 genes, where each gene printed in four replicates in four identical blocks each block of 5×6=30 spots was manufactured on UltraGAPS array slide (Cat. No. 40016, Corning Life Sciences, MA). Each microarray slide carried two identical microarrays for analysis of paired RNA samples in two parallel hybridization reactions, each reaction can be carried out in its own reaction volume (e.g., dual array slide layout).

A paired total RNA, Human Jurkat cells, normal and stimulated by ionomycin/PMA have been purchased from Stratagene (Cat. No. 540111 and 540107). Stimulated cells were incubated 10 hours with 1.5 mM CaCl₂, 4 μM ionomycin and 0.1 mg/L PMA. The quality of total RNA was assessed visually by observing distinct 28S and 18S ribosomal bands. The mRNA was isolated from total RNA samples using Qiagen mRNA Mini Kit (Cat. No. 70022, Qiagen). Two mRNA samples, normal and ionomycin/PMA stimulated, have been hybridized with two identical microarrays of probe oligonucleotides on dual microarray slide as described hereinabove. Each sample consists of about 1 microgram of mRNA diluted in 20 microliters of ExpressHyb hybridization solution (Cat. No. 63683 1, BD Biosciences). Hybridization has been carried out at 37.degree. C. for 12 hours. The microarray dual array slide was subsequently washed in 0.05 M SSC buffer at room temperature for 5 min, followed by rinsing with distilled deionized water, and was dried by centrifugation. To visualize the pattern of hybridized mRNA on the microarray surface the dual array slide was dipped into the solution of 250-nm cationic gold particles (Cat. No. AG14, Sci-Tec, Inc., Knoxville, Tenn.) in 5 mM potassium biphthalate buffer at pH=4.0. The gold-labeled array was scanned by Epson 3200 flatbed scanner operating in dark-mode detection mode.

FIG. 19 shows images of gold-labeled microarray for normal and ionomycin/PMA stimulated Jurkat cell samples and FIG. 20 shows the differential expression pattern generated from images in FIG. 19 by AuroGene 2.20 image acquisition and processing software package (Sci-Tec, Inc, Knoxville, Tenn.). Intra-array reproducibility of the expression pattern has been investigated by examining signals from four replicates for each gene on the microarray, printed in four identical blocks seen in FIG. 19. The expression pattern is highly consistent across four replicates (intra-array consistency). A set of 5 consistently up-regulated and 8 down-regulated genes with fold-change ratio above 3.0 was identified. For this subset of regulated genes a smaller set of 5 genes have been previously reported in a study carried out using NIA-Immunoarray (i.e., for IFNG, JUNB, MYC, NFKB1, and EGR2).

In this example cationic nano-particles in solution at specific solution pH have been used for selective detection of target mRNA hybridized to small synthetic oligonucleotides on microarray surface. Detection was performed without conversion of target mRNA to cDNA and without modification of target mRNA molecules for detection by biotin-streptavidin and antibody-antigen and the like detection systems known in the art.

### EXAMPLE X

### Enzymatic Digestion for Improving Discrimination Sites Carrying Hybridized or Non-Hybridized Molecular Structures

Some applications, such as detection of Single Nucleotide Polymorphisms or identification of extremely low quantity of target species in a sample substance may require advanced discrimination level of sites where probe and target were hybridized vs. the sites with no hybridization. Enhancing discrimination between such sites can be achieved by employing enzymatic digestion of probes, or alternatively probe-target complexes, such that only hybridized, or alternatively only non-hybridized, molecular structures will remain on the substrate and will be detected by labeling with nano-particles as disclosed herein above. In this embodiment of the present invention microarray first hybridized with target molecular structures of a sample substance and after a stringency wash is exposed to a solution containing S1 nuclease isolated from Aspergillus oryzae (Stratagene). The solution can contain from a fraction of 1 to 200 units of the S1 nuclease in a buffer composed of 20-300 mM sodium acetate, 0-5% glycerole (v/v), 0.1-2.8 M NaCl and 0.1-10 mM ZnSO.sub.4. To achieve the desirable effect of degrading unbound probe molecules on the substrate the microarray is incubated in this digestion mix from 1 min to 24 hours at temperature ranging from 15.degree. C. to 45.degree. C. and the solution pH adjusted to the range of about 3 to 10. One skilled in the art will adjust composition of the digestion mix and treatment conditions to achieve satisfactory result. In this example, the S1 nuclease degrades single-stranded nucleic acids on the substrate and efficiently eliminates unbound probes, which otherwise may be a source of false positive identification of hybridized probe-target complexes on the microarray.

We claim:

1. A method of non-specific binding of nano-particles to chemical groups on a substrate surface for detection and quantitation of a latent pattern of target molecular structures on the substrate surface, the method comprising the steps of:
a) creating a latent pattern of target molecular structures on the substrate surface by binding/hybridizing target molecules from a sample substance and probing molecular structures tethered on the substrate surface;
b) preparing a solution of nano-particles, where said nano-particles have Zeta-potential ranging from about minus 150 mV to about minus 1 mV or from about plus 1 mV to about plus 150 mV; or, where said nano-particles carry surface electric charge ranging from about minus 500 mC/m.sup.2 to about minus 3 mC/m.sup.2 or from about plus 3 mC/m.sup.2 to about plus 500 mC/m.sup.2;
c) exposing the substrate surface carrying the latent pattern of target molecular structures to the solution of nano-particles under conditions allowing for the binding of nano-particles to the chemical groups of the substrate surface through a non-specific ionic interaction of nano-particles and chemical groups on the substrate surface, and where said binding yields a layer of bound nano-particles on the substrate surface, the layer of bound particles having a density which varies corresponding to the presence of the target molecular structures on the substrate surface; and
d) measuring the varying density of the bound nano-particles on the substrate surface to determine the location and quantity of the target molecular structures on the substrate surface.

2. The method of claim 1 wherein the nano-particles range in size from about 0.001 .mu.m to about 10 .mu.m and most preferably from about 0.002 .mu.m to about 0.5 .mu.m.

3. The method of claim 1 wherein the nano-particles are materials selected from the group consisting of solid particles and particles of liquid phase.

4. The method of claim 3 wherein the solid particles are materials selected from the group consisting of polymers, metals, metal oxides, carbides, nitrides, borides, chalcogenides, semiconductors, alloys, and mixtures thereof.

5. The method of claim 4 wherein the polymers are materials selected from the group consisting of biologically inert latex consisting of carboxylated styrene butadiene, carboxylated polystyrene, carboxylated polystyrene with amino groups, acrylic acid polymers, methacrylic acid polymers, acrylonitrile butadiene styrene, polyvinyl acetate acrylate, polyvinyl pyridine and vinyl-chloride acrylate.

6. The method of claim 1 wherein the nano-particles are coated with an activation reagent for achieving the desirable surface charge in the range of from about minus 500 mC/m.sup.2 to about minus 3 mC/m.sup.2 or from about plus 3 mC/m.sup.2 to about plus 500 mC/m.sup.2.

7. The method of claim 6 wherein the activation reagent is a material selected from the group consisting of containing an active hydrogen, a nitrile group, a secondary amine group, a primary amine group, trimethylammonium group, or any combination thereof.

8. The method of claim 6 wherein the activation reagent is a material selected from the group consisting of cationic, anionic, and zwitterionic detergents, bile acid salts, or any combination thereof.

9. The method of claim 1 wherein the latent pattern of target molecular structures is formed by hybridized nucleic acids and where the nano-particles have Zeta-potential in the range of from about plus 1 mV to about plus 150 mV or where said nano-particles carry surface electric charge in the range of from about plus 3 mC/m.sup.2 to about plus 500 mC/m.sup.2.

10. The method of claim 1 wherein the latent pattern of target molecular structures results from specific binding of target and probe proteins.

11. The method of claim 10, wherein the latent pattern of target molecular structures is treated in a solution of anionic detergent and subsequently is exposed to the solution of nano-particles having Zeta-potential in the range of from about plus 1 mV to about plus 150 mV or where said nano-particles carry surface electric charge in the range of from about plus 3 mC/m.sup.2 to about plus 500 mC/m.sup.2.

12. The method of claim 11 wherein the anionic detergent is a material selected from the group consisting of Chenodeoxycholic acid; Chenodeoxycholic acid sodium salt; Dehydrocholic acid; Deoxycholic acid; Deoxycholic acid; Deoxycholic acid methyl ester; Digitonin; Digitoxigenin; N;N-Dimethyldodecylamine N-oxide; Docusate sodium salt waxy solid; Docusate sodium salt; Glycochenodeoxycholic acid sodium salt; Glycocholic acid hydrate; Glycocholic acid sodium salt hydrate; Glycodeoxycholic acid monohydrate; Glycodeoxycholic acid sodium salt; Glycodeoxycholic acid sodium salt; Glycolithocholic acid 3-sulfate disodium salt; Glycolithocholic acid ethyl ester; N-Lauroylsarcosine sodium salt, N-Lauroylsarcosine sodium salt; N-Lauroylsarcosine solution; N-Lauroylsarcosine solution; Lithium dodecyl sulfate; Lugol solution; Niaproof4; Niaproof 4; Triton QS-15; Triton QS-44; 1-Octanesulfonic acid sodium salt; 1-Octanesulfonic acid sodium salt, Sodium 1-butanesulfonate; Sodium 1-ecanesulfonate; Sodium 1-decanesulfonate; Sodium 1-dodecanesulfonate; Sodium 1-heptanesulfonate anhydrous; Sodium 1-heptanesulfonate anhydrous; Sodium 1-nonanesulfonate; Sodium 1-propanesulfonate monohydrate; Sodium 2-bromoethanesulfonate; Sodium cholate hydrat; Sodium choleate; Sodium deoxycholate; Sodium deoxycholate monohydrate; Sodium dodecyl sulfate; Sodium hexanesulfonate; Sodium octyl sulfate; Sodium pentanesulfonate; Sodium taurocholate; Taurochenodeoxycholic acid sodium salt; Taurodeoxycholic acid sodium salt monohydrate; Taurohyodeoxycholic acid sodium salt hydrate; Taurolithocholic acid 3-sulfate disodium salt; Tauroursodeoxycholic acid sodium salt; Triton X-200 solution; Triton® XQS-20 solution; Trizma® dodecyl sulfate; Ursodeoxycholic acid, and mixtures thereof.

13. The method of claim 1 further comprising the step of treating the substrate surface and latent pattern of molecular structures with a solution of a positively charged natural or synthetic polymer material selected from the group consisting of substances containing an active hydrogen, e.g., a nitrile group, a secondary amine group, a primary amine group, trimethylammonium group, or any combination thereof.

14. The method of claim 1 wherein the latent pattern of target molecular structures results from enzymatic digestion of hybridized/bound molecular structures on the substrate surface.

15. A method of non-specific ionic binding of nano-particle powder to chemical groups on a substrate surface for detection and quantitation of a latent pattern of target molecular structures on the substrate surface, the method comprising the steps of:
a) creating a latent pattern of target molecular structures on the substrate surface by binding/hybridizing target molecules from a sample substance and probe molecular structures tethered on the surface of the solid substrate;
b) preparing nano-particle powder, where at least some nano-particles carry surface electric charges ranging from about minus 500 mC/m.sup.2 to about minus 3 mC/m.sup.2 or from about plus 3 mC/m.sup.2 to about plus 500 mC/m.sup.2;
c) exposing the substrate surface carrying the latent pattern of target molecular structures to the powder of nano-particles under conditions allowing for the binding of nano-particles to the chemical groups on the substrate surface through a non-specific ionic interaction of nano-particles and chemical groups on the substrate surface, and where said binding yields a layer of bound nano-particles on the substrate surface, the layer of bound particles having a density which varies corresponding to the presence of the target molecular structures on the substrate surface; and
d) measuring the varying density of the bound nano-particles on the substrate surface to determine the location and quantity of the target molecular structures on the substrate surface.

16. A method of preparing a solution of nano-particles for detection and quantitation of a latent pattern of target molecular structures on a surface of solid support, the method comprising the steps of:
a) preparing a solution of nano-particles and activation reagent selected from the group consisting of surfactants, waxes, oils, silys, synthetic and natural polymers, resins and mixtures thereof;
b) incubating the solution of nano-particles and the activation reagent for a period of time from about 1 sec to about 24 hours at temperature in the range of from about 4.degree.C. to about 95.degree.C.:
c) if required, removing unbound activation reagent from the solution by centrifuging the solution of nano-particles and activation reagent and by discarding the natant or by chemically neutralizing the activation reagent;
d) adjusting the concentration, pH and ionic strength of the solution of activated nano-particles by adding a buffer solution at desirable ionic strength and pH to adjust the ionic strength of the solution most preferably to the range of from about 0.001 mM of buffer ions to about 100 mM of buffer ions and solution pH to the range of from about pH=3.0 to about pH=9.0.

17. The method of claim 16 wherein nano-particles are materials selected from the group consisting of solid particles and particles of liquid phase.

18. The method of claim 17 wherein particles of liquid phase essentially consist of emulsions.

19. The method of claim 17 wherein the solid particles essentially consist of materials selected from the group consisting of polymers, metals, metal oxides, carbides, nitrides, borides, chalcogenides, semiconductors, alloys, and mixtures thereof.

20. The method of claim 19 wherein the polymers essentially consist of materials the group consisting of biologically inert latex consisting of carboxylated styrene butadiene, carboxylated polystyrene, carboxylated polystyrene with amino groups, acrylic acid polymers, methacrylic acid polymers, acrylonitrile butadiene styrene, polyvinyl acetate acrylate, polyvinyl pyridine and vinyl-chloride acrylate.

21. The method of claim 16 wherein the activation reagent essentially consists of material selected from the group consisting of materials containing an active hydrogen, e.g., —COOH, —CONH.sub.2, a nitrile group, a secondary amine group, a primary amine group, trimethylammonium group, or any combination thereof.

22. The method of claim 16 wherein the activation reagent is selected from the group of materials consisting of cationic, anionic, and zwitterionic detergents, bile acid salts, or any combination thereof.

23. A method of preparing powder of nano-particles for detection and quantitation of a latent pattern of target molecular structures on a surface of solid support, the method comprising the steps of:
a) exposing nano-particles to an activation reagent selected from the group consisting of surfactants, waxes, oils, silys, synthetic and natural polymers, resins and mixtures thereof; said exposing nano-particles to activation reagent can be carried in solution or by exposing the particles to activation reagent(s) in a gas phase;
b) incubating the solution of nano-particles and activation reagent for a period of time from about 1 sec to about 24 hours at temperature in the range of from about 4.degree.C. to about 95.degree.C.; or by incubating particles in presence of activation reagent in gas phase for period of time from about 1 min to about 24 hours;
c) isolating nano-particles from solution by centrifugation and discarding the natant:
d) drying the isolated nano-particle substance and reconstituting by milling the substance consisting from nano-particles to nano-particle powder.

24. The method of claim 23 wherein the nano-particles comprise solid particles selected from the group consisting of polymers, metals, metal oxides, carbides, nitrides, borides, chalcogenides, semiconductors, alloys, and mixtures thereof.

25. The method of claim 24 wherein the polymers essentially consist of material selected from the group of biologically inert latex consisting essentially of carboxylated styrene butadiene, carboxylated polystyrene, carboxylated polystyrene with amino groups, acrylic acid polymers, methacrylic acid polymers, acrylonitrile butadiene styrene, polyvinyl acetate acrylate, polyvinyl pyridine vinyl-chloride acrylate, and mixtures thereof.

26. The method of claim 23 wherein the activation reagent is material selected from the group consisting of substances containing an active hydrogen, a nitrile group, a secondary amine group, a primary amine group, trimethylammonium group, or any combination thereof.

27. The method of claim 23 wherein the activation reagent is material selected from the group of substances consisting essentially of cationic, anionic, and zwitterionic detergents, bile acid salts, or any combination thereof.

28. A kit for detecting quantitation of a latent pattern of target molecular structures on a substrate surface according to the method of claim 1, the kit comprising multiple containers having appropriate amounts of reagents, including some or all of the following: a) a container containing a suitable colloidal solution or powder; b) a container containing an activating solution; c) a container containing a buffer solution for preparing solution of nano-particles at desirable pH and ionic strength; d) a container containing solution of polymer substance for blocking the substrate prior to development in a colloidal solution or exposing the substrate to powder of nano-particles; e) a container or attachable chamber suitable to carry out hybridization or a binding reaction: and f) a container suitable for washing the substrate by dipping in or rinsing with a washing buffer.

29. A kit for detecting quantitation of a latent pattern of target molecular structures on a substrate surface according to the method of claim 15, the kit comprising multiple containers having appropriate amounts of reagents, including some or all of the following: a) a container containing a suitable powder of nano-particles; b) a container containing solution of polymer substance for blocking the substrate prior to exposing the substrate to the powder of nano-particles; e) a container or attachable chamber suitable to exposing the substrate to powder of nano-particles; and f) a container suitable for washing the substrate by dipping in or rinsing with a washing buffer.
